(19) United States
(12) Patent Application Publication
(10) Pub. No.: US 2001/0046680 A1
(43) Pub. Date: Nov. 29, 2001

(54) Identification of polypeptides and nucleic acid molecules using linkage between DNA and polypeptide

(76) Inventor: Zhongping Yu, San Diego, CA (US)

Correspondence Address: DAVID R PRESTON & ASSOCIATES, 12625 HIGH BLUFF DRIVE, SUITE 205, SAN DIEGO, CA, 92130, US

(21) Appl. No.: 09/849,020

(22) Filed: May 4, 2001

Related U.S. Application Data
(63) Continuation-in-part of application No. 09/821,160, filed on Mar. 29, 2001.
(60) Provisional application No. 60/156,990, filed on Oct. 1, 1999.
(60) Provisional application No. 60/178,420, filed on Jan. 27, 2000.
(60) Provisional application No. 60/202,066, filed on May 5, 2000.
(60) Provisional application No. 60/226,535, filed on Aug. 16, 2000.

(30) Foreign Application Priority Data
Sep. 27, 2000 (US) PCT/US00/26511

(51) Int. Cl. C12Q 1/68; C07H 21/02; C07H 21/04

(52) U.S. Cl. 435/6; 536/23.1

(57) ABSTRACT

The present invention provides efficient methods for identifying nucleic acid molecules and polypeptides encoded thereby. These methods can be performed using transcription and translation systems or methods, or using translation methods. The result of these methods is a peptide or polypeptide that binds its own encoding nucleic acid molecule. The invention includes nucleic acid molecules that comprise an open reading frame, and that comprise linking moieties that can link the nucleic acid molecules to the polypeptides they encode. The invention methods of identifying test compounds and targets using these nucleic acid molecules.

This application claims priority to and incorporates by reference U.S. provisional application No. 60/156,990 which was filed Oct. 1, 1999; U.S. provisional application No. 60/178,420 which was filed Jan. 27, 2000; U.S. provisional application No. 60/202,066 which was filed May 5, 2000; U.S. provisional application No. 60/226,535, filed Aug. 16, 2000; U.S. application Ser. No. 09/821,160, filed Mar. 39, 2001; and PCT application PCT/US00/26511, filed Sep. 27, 2000.

### TECHNICAL FIELD

The present invention relates generally to the fields of molecular biology, in particular to methods for identifying nucleic acid molecules and polypeptides.

### BACKGROUND

Efforts to identify polypeptides that have biological activity such as enzymatic activity or binding activity and the nucleic acid molecules that encode the polypeptide have utilized a variety of methods. Such methods include genomics and combinatorial biology.

Genomics generally identifies nucleic acid molecules within a genome, often without regard to the function of the nucleic acid molecule or the polypeptide encoded thereby. Genomics tends to provide information as to the sequence or partial sequence of a nucleic acid molecule but does not provide significant information as to the function of the nucleic acid sequence or the polypeptide encoded thereby. The outcome of genomics is generally the identification of expression sequence tags (ESTs) or the trapping of promoters or genes. Functional genomics generally attempts to contemporaneously identify a gene and its function. Functional genomics relies on the use of cell-based or organism-based assay systems or comparative analyses, which tend to be cumbersome, complicated, time-consuming and expensive.

Combinatorial biology generally identifies nucleic acid sequences and polypeptides encoded thereby that are not isolated from a biological source but nonetheless have a biological activity. Combinatorial biology provides random or semi-random groups (or libraries) of nucleic acid molecules or polypeptides. The libraries are screened for an activity, such as a binding activity. One method of combinatorial biology, known as SELEX, relies on the folding of RNA molecules to provide an RNA molecule that has receptor-ligand binding capabilities. This type of receptor-ligand binding, though interesting, is a rather rare event in cellular processes.

Another method of combinatorial biology provides a library of bacteriophages that display a variety of random polypeptides on their surface. The genome of the bacteriophage includes the nucleic acid molecule that encodes the random polypeptide displayed on the surface. The binding of a phage to a receptor during “panning” procedure results in the isolation of a bacteriophage that includes the random polypeptide and the encoding nucleic acid molecule. This type of combinatorial biology results in the identification of interesting polypeptides and nucleic acid sequences, but the methods used rely on complex in vivo biological processes to produce bacteriophage. These complex processes tend to reduce the complexity of the combinatorial biology libraries and make these methods not particularly suitable for automation.

In vitro combinatorial biology methods have also been used. For example, random nucleic acid sequence can be made part of an RNA molecule that is translated by a plurality of ribosomes to form a polysome. The polysome structure can be “stalled” such that the RNA molecule is attached to the ribosomes and a partially translated polypeptide. This stalling can be accomplished using a variety of methods, such as lowering the temperature and adding chemicals to stabilize the polypeptide-ribosome-RNA ternary structure. The stalled polysomes structures can then be panned for binding to a ligand to identify polysomes that include random nucleic acid molecules that encode polypeptides that can bind with a ligand. Another method for in vitro combinatorial biology methods is using RNA-protein fusions. This method relies on incorporation of puromycin that is ligated to the 3′-end of a messenger RNA into the C-terminal of a polypeptide by a ribosome. The RNA and polypeptide are linked or “fused” by a covalent bond. The ribosome is then dissociated from the translational machinery and the RNA-polypeptide fusion can be screened against target molecules. The RNA on the selected RNA-polypeptide fusion can be enriched by reverse-transcription polymerase chain reaction (PCR). However, ligating puromycin to the 3′-end of RNA is not efficient and RNA is easily degraded in experiment. More importantly, both of these methods require a separate step to transcribe the DNA template and subsequently purify the RNA transcript for translation reaction. Therefore, it is cumbersome to perform the experiment and the entire procedures are very difficult to streamlined and automated.

The present invention provided methods and articles of manufacture that address the problems associated with combinatorial biology. The present invention provides related benefits as well.

### BRIEF DESCRIPTION OF THE FIGURES

FIG. 1 illustrates one aspect of structural features of the DNA construct in a transcription ternary complex.

FIG. 2 illustrates one aspect of structural features of the DNA construct in a transcription ternary complex

FIG. 3 illustrates one aspect of structural features of the DNA construct in a transcription ternary complex.

FIG. 4 illustrates one aspect of structural features of the DNA construct in a transcription ternary complex.

FIG. 5 illustrates one method of linking a DNA molecule to its owning encoding peptide and selecting the desired DNA-polypeptide complexes.

FIG. 6 illustrates one aspect of making a DNA-polypeptide complex from natural mRNA and selecting desired DNA-polypeptide complexes.

FIG. 7 depicts one structural aspect of a single-stranded DNA molecule of the present invention.

FIG. 8 depicts one structural aspect of a single-stranded DNA molecule of the present invention.

FIG. 9 depicts a schematic diagram of one aspect of the present invention.

FIG. 10 depicts a schematic diagram of one aspect of the present invention.

FIG. 11 illustrates one aspect of making a DNA-polypeptide complex from natural mRNA and selecting desired DNA-polypeptide complexes.

### SUMMARY

The present invention provides compositions and efficient methods for identifying nucleic acids and peptides and polypeptides encoded thereby. These methods are performed using translation systems and methods, or using transcription and translation systems or methods. When transcription and translation methods are used, the transcription and translation reactions may or may not be coupled. The result of these methods is a complex that includes a polypeptide that is covalently or non-covalently linked with its own encoding nucleic acid molecule. The nucleic acid molecule can comprise a moiety that links the nucleic acid molecule to its own encoded polypeptide.

A first aspect of the present invention is a nucleic acid molecule that comprises a transcription regulatory region, a transcription termination moiety, and, preferably, a linking moiety that can directly or indirectly link the nucleic acid molecule with a peptide, and encodes an open reading frame for a peptide or polypeptide. The nucleic acid molecule can optionally encode a ribosome binding RNA sequence. The nucleic acid molecule can be provided in a vector.

A second aspect of the present invention is a library of nucleic acid molecules of the first aspect of present invention, either alone, linked with their own encoded polypeptides or with a substance of interest or as part of a vector.

A third aspect of the present invention is a method of linking a nucleic acid molecule of the present invention to a peptide that is encoded by the nucleic acid molecule. The method includes: transcribing at least a portion of a nucleic acid molecule of the present invention using at least one RNA polymerase, such that at least one RNA polymerase terminates at a transcription termination site so that transcription elongation ternary complexes, which comprise a nucleic acid molecule template, an RNA transcript, and RNA polymerase, are formed. Translation systems are employed that can translate, at least in part, the RNA template in the complex. The peptide or polypeptide translated from the RNA template can preferably bind or be coupled to a linking moiety that is bound by the nucleic acid molecule. The result of these procedures is a peptide or polypeptide that is directly or indirectly bound to the nucleic acid molecule that encodes the polypeptide.

A fourth aspect of the present invention is a method for identifying a nucleic acid molecule using the methods of the second aspect of the present invention.

A fifth aspect of the present invention is a method for identifying a polypeptide using the methods of the second aspect of the present invention.

A sixth aspect of the present invention is a DNA molecule that 1) comprises a linking moiety and 2) encodes an open reading frame for a peptide or polypeptide. Also, the DNA molecule can contain a ribosome binding sequence (RBS).

A seventh aspect of the present invention is a library of DNA molecules of the sixth aspect of the present invention, either alone, linked with their own encoded polypeptides, or with a substance of interest or as part of a vector.

An eighth aspect of the present invention is a method for of linking a DNA molecule of the seventh aspect of the present invention to a peptide that is encoded by at least a portion of the nucleic acid molecule. The method includes: translating a DNA that comprises an open reading frame and a linking moiety or binding region so that complexes that comprise the polypeptide encoded by the open reading frame become bound to the DNA molecule. The result of these procedures is a peptide or polypeptide that is directly or indirectly bound to the nucleic acid molecule that encodes the polypeptide.

A ninth aspect of the present invention is a method for identifying a DNA molecule using the methods of the eighth aspect of the present invention.

A tenth aspect of the present invention is a method for identifying a polypeptide using the methods of the eighth aspect of the present invention.

An eleventh aspect of the present invention is methods of identifying test compounds using the methods present invention and test compounds and pharmaceutical compositions identified by such methods.

A twelfth aspect aspect of the present invention is methods of identifying targets using the methods present invention and targets identified by such methods.

### DETAILED DESCRIPTION OF THE INVENTION

Definitions

Unless defined otherwise, all technical and scientific terms used herein have the same meaning as commonly understood by one of ordinary skill in the art to which this invention belongs. Generally, the nomenclature used herein and the laboratory procedures in cell culture, chemistry, microbiology, molecular biology, and cell biology and cell culture described below are well known and commonly employed in the art. Conventional methods are used for these procedures, such as those provided in the art and various general references (Sambrook et al., Molecular Cloning: A Laboratory Manual, 2nd edition, Cold Spring Harbor Press, Cold Spring Harbor, N.Y. (1989); and Harlow and Lane, Antibodies, A Laboratory Manual, Cold Spring Harbor Press (1988)). Where a term is provided in the singular, the inventors also contemplate the plural of that term. The nomenclature used herein and the laboratory procedures described below are those well known and commonly employed in the art. As employed throughout the disclosure, the following terms, unless otherwise indicated, shall be understood to have the following meanings:

“Membrane permeable derivative” refers to a chemical derivative of a compound that increases membrane permeability of the compound. These derivatives are made better able to cross cell membranes because hydrophilic groups are masked to provide more hydrophobic derivatives. Also, the permeability-making groups can be designed to be cleaved from the compound within a cell to make the compound more hydrophilic once within the cell. Because the substrate is more hydrophilic than the membrane permeate derivative, it preferentially localizes within the cell (U.S. Pat. No. 5,741,657 to Tsien et al., issued Apr. 21, 1998).

“Isolated polynucleotide” refers to a polynucleotide of genomic, cDNA, or synthetic origin, or some combination thereof, which by virtue of its origin, the isolated polynucleotide (1) is not associated with the cell in which the isolated polynucleotide is found in nature, or (2) is operably linked to a polynucleotide that it is not linked to in nature. The isolated polynucleotide can optionally be linked to promoters, enhancers, or other regulatory sequences.

“Isolated protein” refers to a protein of cDNA, RNA derived from cDNA, DNA, RNA or synthetic origin, or some combination thereof, which by virtue of its origin the isolated protein (1) is not associated with proteins normally found within nature, or (2) is isolated from the cell in which it normally occurs, or (3) is isolated free of other proteins from the same cellular source (for example, free of cellular proteins), or (4) is expressed by a cell from a different species, or (5) does not occur in nature.

“Peptide” is a sequence of two or more amino acids joined by peptide bonds. Peptides can include other moieties, such as chemical groups, drug molecules, detectable labels, or specific binding members that are reversibly or irreversibly bound to one or more amino acids of the peptide.

“Polypeptide” is used herein as a generic term to refer to protein or fragments or analogs of a protein.

“Active fragment” refers to a fragment of a parent molecule, such as an organic molecule, nucleic acid molecule, or protein or polypeptide, or combinations thereof, that retains at least one activity of the parent molecule.

“Naturally occurring” refers to the fact that an object can be found in nature. For example, a polypeptide or polynucleotide sequence that is present in an organism, including viruses, that can be isolated from a source in nature and which has not been intentionally modified by man in the laboratory is naturally occurring.

“Operably linked” refers to a juxtaposition wherein the components so described are in a relationship permitting them to function in their intended manner. A control sequence operably linked to a coding sequence is ligated in such a way that expression of the coding sequence is achieved under conditions compatible with the control sequences.

“Control sequences” refers to polynucleotide sequences that effect the expression of coding and non-coding sequences to which they are operably linked. When the control sequences are used to control the transcription of DNA template, it is also called a transcription regulatory region. The nature of such control sequences differs depending upon the host organisms and enzymes; in prokaryotes, such control sequences generally include promoter, ribosomal binding site, and translation initiation and termination codons; in eukaryotes, generally, such control sequences include promoters and translation initiation and termination sequences. The term control sequences is intended to include components whose presence can influence expression, and can also include additional components whose presence is advantageous, for example, leader sequences and fusion partner sequences.

A “transcription regulatory region” is a region of a nucleic acid that controls the transcription of a nucleic acid sequence to which it is operably linked.

A “ribosome binding site” or “ribosome binding sequence” or “RBS” is a nucleotide sequence that allows the binding of the ribosome to a nucleic acid molecule. Ribosome binding sites known in the art that allow for ribosome binding and the initiation of translation are, for example, Shine-Dalgarno sequences, Kozak sequences, and IRES sequences. As used herein, Shine-Dalgorno sequences and Kozak sequences can be identified canonical sequences, or substantially homologous sequences that can be bound by ribosomes and thereby initiate translation. IRES sequences can be those already identified, or any identified in the future, such as by functional assay. A “ribosome RNA binding sequence” specifies that the nucleotide sequence consists of or essentially consists of an RNA sequence.

“Nucleic acid” or “nucleic acid molecule” or “polynucleotide” refers to a polymeric form of nucleotides of a least ten bases in length. A nucleic acid molecule can be DNA, RNA, or a combination of both. A nucleic acid molecule can also include sugars other than ribose and deoxyribose incorporated into the backbone, and thus can be other than DNA or RNA. A nucleic acid can comprise nucleobases that are naturally occurring or that do not occur in nature, such as xanthine, derivatives of nucleobases such as 2-aminoadenine and the like. A nucleic acid molecule of the present invention can have linkages other than phosphodiester linkages. A nucleic acid molecule can also be a peptide nucleic acid molecule (PNA) or can comprise PNA residues. A nucleic acid molecule can be of any length, and can be single-stranded or double-stranded, or partially single-stranded and partially double-stranded. A nucleic acid molecule can comprise other entities, such as drug molecules, detectable labels, linking moieties, or specific binding members.

“Directly” in the context of a biological process or processes, refers to direct causation of a process that does not require intermediate steps, usually caused by one molecule contacting or binding to another molecule (the same type or different type of molecule). For example, molecule A contacts molecule B, which can cause molecule B to exert effect X that is part of a biological process. In terms of binding, “directly” means that molecule A contacts and binds molecule B without intermediate molecules that mediate the binding.

“Indirectly” in the context of a biological process or processes, refers to indirect causation that requires intermediate steps, usually caused by two or more direct steps. For example, molecule A contacts molecule B to exert effect X which in turn causes effect Y. In terms of binding, “indirectly” means that molecule A binds molecule B by contacting at least one intermediate molecule that mediates the binding.

“Sequence homology” refers to the proportion of base matches between two nucleic acid sequences or the proportion of amino acid matches between two amino acid sequences. When sequence homology is expressed as a percentage, for example 50%, the percentage denotes the proportion of matches of the length of sequences from a desired sequence that is compared to some other sequence. Gaps (in either of the two sequences) are permitted to maximize matching; gap lengths of 15 bases or less are usually used, 6 bases or less are preferred with 2 bases or less more preferred. When using oligonucleotides as probes or treatments, the sequence homology between the target nucleic acid and the oligonucleotide sequence is generally not less than 17 target base matches out of 20 possible oligonucleotide base pair matches (85%); preferably not less than 9 matches out of 10 possible base pair matches (90%), and most preferably not less than 19 matches out of 20 possible base pair matches (95%).

“Corresponds to” refers to a polynucleotide sequence that is homologous (for example is identical, not strictly evolutionarily related) to all or a portion of a reference polynucleotide sequence, or to a polypeptide sequence that is identical to all or a portion of a reference polypeptide sequence. In contradistinction, the term “complementary to” is used herein to mean that the complementary sequence will base pair with all or a portion of a reference polynucleotide sequence. For illustration, the nucleotide sequence TATAC corresponds to a reference sequence TATAC and is complementary to a reference sequence GTATA.

“Conservative amino acid substitutions” refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine and tryptophan; a group of amino acids having basic side chains is lysine, arginine and histidine; a group of amino acids having acidic side chains is aspartic acid and glutamic acid; and a group of amino acids having sulfur-containing side chain is cysteine and methionine. Preferred conservative amino acid substitution groups are: valine-leucine-isoleucine; phenylalanine-tyrosine; lysine-arginine; alanine-valine; glutamic acid-aspartic acid; and asparagine-glutamine.

“Test compound” refers to a chemical, compound, composition or extract to be tested by at least one method of the present invention for at least one activity for at least one activity such as putative modulation of a biological process or specific binding capability. Test compounds can include small molecules, drugs, proteins or peptides or active fragments thereof, such as antibodies or fragments or active fragments thereof, nucleic acid molecules such as DNA, RNA or combinations thereof, antisense molecules or ribozymes, or other organic or inorganic molecules, such as lipids, carbohydrates, or any combinations thereof. Test compounds that include nucleic acid molecules can be provided in a vector, such as a viral vector, such as a retrovirus, adenovirus or adeno-associated virus, a liposome, a plasmid or with a lipofection agent. Test compounds, once identified, can be agonists, antagonists, partial agonists or inverse agonists of a target. A test compound is usually not known to bind to the target of interest.

“Control test compound” refers to a compound known to bind to the target (for example, a known agonist, antagonist, partial agonist or inverse agonist). Test compound does not typically include a compound added to a mixture as a control condition that alters the function of the target to determine signal specificity in an assay. Such control compounds or conditions include chemicals that (1) non-specifically or substantially disrupt protein structure (for example denaturing agents such as urea or guandium, sulfhydryl reagents such as dithiothreitol and beta-mercaptoethanol), (2) generally inhibit cell metabolism (for example mitochondrial uncouplers) and (3) non-specifically disrupt electrostatic or hydrophobic interactions of a protein (for example, high salt concentrations or detergents at concentrations sufficient to non-specifically disrupt hydrophobic or electrostatic interactions). The term test compound also does not typically include compounds known to be unsuitable for a therapeutic use for a particular indication due to toxicity to the subject. Usually, various predetermined concentrations of test compounds are used for determining their activity. If the molecular weight of a test chemical is known, the following ranges of concentrations can be used: between about 0.001 micromolar and about 10 millimolar, preferably between about 0.01 micromolar and about 1 millimolar, more preferably between about 0.1 micromolar and about 100 micromolar. When extracts are uses a test compounds, the concentration of test chemical used can be expressed on a weight to volume basis. Under these circumstances, the following ranges of concentrations can be used: between about 0.001 micrograms/ml and about 1 milligram/ml, preferably between about 0.01 micrograms/ml and about 100 micrograms/ml, and more preferably between about 0.1 micrograms/ml and about 10 micrograms/ml.

“Target” refers to a biochemical entity involved in a biological process. Targets are typically proteins that play a useful role in the physiology or biology of an organism. A therapeutic composition or compound typically binds to a target to alter or modulate its function. As used herein, targets can include, but not be limited to, cell surface receptors, G-proteins, G-protein coupled receptors, kinases, phosphatases, ion channels, lipases, phosholipases, nuclear receptors, transcription factors, intracellular structures, tubules, tubulin, antibodies and the like.

A “therapeutic target” or a “pharmaceutical target” is a target that when modulated can have a therapeutic effect.

A “purification target” is a target that is useful in purification schemes, such as, for example, regions of antibodies such as the Fc region.

A “diagnostic target” is a target that is useful in diagnostics, such as cell surface epitopes or markers on etiological agents.

“Label” or “labeled” refers to incorporation of a marker which may or may not be used for detection purpose. For example by incorporation of a radiolabled compound or attachment to a polypeptide of moieties such as biotin that can be detected by the binding of a section moiety, such as marked avidin. On the other hand, if a protein is labeled by a biotin, the protein can be attached to a nucleic acid that is labeled with an avidin. Thus, the protein and nucleic acid can form a complex. Various methods of labeling polypeptide, nucleic acids, carbohydrates, and other biological or organic molecules are known in the art. Such labels can have a variety of readouts, such as radioactivity, fluorescence, color, chemiluminescence or other readouts known in the art or later developed. The readouts can be based on enzymatic activity, such as beta-galactosidase, beta-lactamase, horseradish peroxidase, alkaline phosphatase, luciferase; radioisotopes such as ³H, ¹⁴C, ³⁵S, ³²P, ¹²⁵I or ¹³¹I); fluorescent proteins, such as green fluorescent proteins; or other fluorescent labels, such as FITC, rhodamine, and lanthanides. Where appropriate, these labels can be the product of the expression of reporter genes, as that term is understood in the art. Examples of reporter genes are beta-lactamase (U.S. Pat. No. 5,741,657 to Tsien et al., issued Apr. 21, 1998) and green fluorescent protein (U.S. Pat. No. 5,777,079 to Tsien et al., issued Jul. 7, 1998; U.S. Pat. No. 5,804,387 to Cormack et al., issued September 8, 1998).

“Specific binding member” is one of two different molecules having an area on the surface or in a cavity which specifically binds to and is thereby defined as complementary with a particular spatial and polar organization of the other molecule. A specific binding member can be a member of an immunological pair such as antigen-antibody, biotin-avidin, hormone-hormone receptor, nucleic acid duplexes, IgG-protein A, DNA-DNA, DNA-RNA, and the like.

“Substantially pure” refers to an object species or activity that is the predominant species or activity present (for example on a molar basis it is more abundant than any other individual species or activities in the composition) and preferably a substantially purified fraction is a composition wherein the object species or activity comprises at least about 50 percent (on a molar, weight or activity basis) of all macromolecules or activities present. Generally, as substantially pure composition will comprise more than about 80 percent of all macromolecular species or activities present in a composition, more preferably more than about 85%, 90%, 95% and 99%. Most preferably, the object species or activity is purified to essential homogeneity, wherein contaminant species or activities cannot be detected by conventional detection methods) wherein the composition consists essentially of a single macromolecular species or activity. The inventors recognize that an activity may be caused, directly or indirectly, by a single species or a plurality of species within a composition, particularly with extracts.

“Pharmaceutical agent or drug” refers to a chemical, composition or activity capable of inducing a desired therapeutic effect when property administered by an appropriate dose, regime, route of administration, time and delivery modality.

“Sample” means any biological sample, preferably derived from a test animal, such as a mouse, rat, rabbit or monkey, or a patient, such as a human. Samples can be from any tissue or fluid, such as neural tissues, central nervous tissues, internal organs such as pancreas, liver, lung, kidney, muscle, skeletal muscle, urine, feces, blood, fluids from body cavities or the central nervous system, or samples from various body cavities such as the mouth or nose. Samples derived from urine and feces contain cells of the immunological, urinary or digestive tract and can be a rich source of sample. Such samples can be obtained using methods known in the art, such as biopsies, aspirations, scrapings or simple collection. A sample can be taken from a test animal or patient that is either living or dead.

“Ribozyme” means enzymatic RNA molecules capable of catalyzing the specific cleavage of RNA. The mechanism of ribozyme action involves sequence-specific hybridization of the ribozyme molecule to complementary target RNA, followed by endonucleolytic cleavage.

A “DNA” molecule refers to either single- or double-stranded deoxyribonucleic acid. A DNA molecule can include nucleotide analogues or derivatives, such as dideoxynucleotides, or nucleotides comprising non-naturally occurring bases, such as inosine, and can comprise one or more linkages other than phosphodiester linkages, such as for example, phosphoramide or phosphothioate linkages. A DNA molecule can also comprise other chemical moieties, such as labels, specific binding members, and linking moieties.

A “moiety” refers to any chemical or biochemical structure. Preferably, a moiety is a structure that can be incorporated into, or covalently or noncovalently, reversibly or irreversibly bound to nucleic acid, polypeptide, or both.

A “transcription termination moiety” refers to a region or structure of a nucleic acid molecule, a protein or polypeptide, or a compound or any other entity or moiety, that is directly or indirectly linked to a nucleic acid molecule and impedes with the progress of, stalls, or stops the functional migration of an RNA polymerase along the nucleic acid template.

A “linking moiety” refers to an entity that is capable of directly or indirectly linking a peptide or polypeptide to a nucleic acid molecule, such as its own encoding nucleic acid molecule. A linking moiety can be a compound or an entity directly or indirectly linked to a nucleic acid. For example, a puromycin molecule bound to a DNA molecule can be a linking moiety that can be incorporated to the polypeptide encoded by the DNA molecule by a ribosome. Other nonlimiting examples of linking moieties are peptide nucleic acid sequences, peptide sequences, nucleic acid sequences, nitrilotriacetic acid, digoxigenin, and biotin, all of which can optionally but preferably be covalently bound to a nucleic acid molecule of the present invention.

“Link” refers to any direct or indirect interactions between two molecules, which includes covalent bonds, coordinate bonds, hydrophobic interaction, electrostatic interaction, or a combination thereof.

A “random sequence” refers to a fully random, partially random, or semi-random sequence of nucleic acid bases that forms a nucleic acid molecule or amino acids that form a polypeptide. Random sequences can be made using synthetic methods as they are known in the art, such as solid phase nucleic acid or solid phase polypeptide synthesis, or by enzymatic methods, such as polymerase reactions or digesting polypeptides or nucleic acids of natural or synthetic origin to obtain fragments thereof, or by any combination of these methods. Fully random refers to 1) sequences that have been made without statistical weight to the probability of inserting any one of the set of naturally-occurring bases or amino acids at a given position of the random sequence, or 2) sequences that have been made by fragmentation of at least one nucleic acid molecule. Semi-random refers to sequences that have been made with statistical weight as bases/amino acids and/or their sequence and can be made using synthetic methods known in the art or by digesting polypeptides or nucleic acid molecules (see, U.S. Pat. No. 5,270,163 to Gold et al., issued Dec. 14, 1993; and U.S. Pat. No. 5,747,253 to Ecker et al., issued May 5, 1998). Semi-random sequences can be nucleic acid or amino acid sequences that have been synthesized such that particular sequence combinations are preferred over other sequence combinations. For example, a semi-random nucleic acid sequence can be biased to preferentially include only a subset of the nucleic acid codons that encode particular amino acids, or can be biased such that the frequency of stop codons in the sequence is reduced. Similarly, a semi-random nucleic acid sequence can be synthesized such that, for example, codons for hydrophobic amino acids are less abundant in the sequence than would occur if the sequence were totally random. Semi-random sequences can be made by directed chemical synthesis, and can, for example, be based on the synthesis of preferred codons that can be built into a multi-codon sequence as disclosed in PCT application US99/22436 (WO 00/18778) to Lohse et al., published Apr. 6, 2000, which is herein incorporated by reference. Partially random sequences are sequences that are in part known or identified sequences and are in part fully random or partially random sequences, and can also be made by modifying or adding to identified or fixed sequences (Pasqualini and Ruoslahti, Nature 380:364-366 (1999); and U.S. Pat. No. 5,270,163 to Gold et al., issued Dec. 14, 1993).

A “sequence of interest” refers to a nucleic acid sequence or nucleic acid molecule that has been selected for by screening or otherwise identified. A sequence of interest can also be at least a portion of a known nucleic acid molecule or nucleic acid sequence. Preferably, an activity, such as an enzymatic activity or binding activity, of the amino acid sequence that can be partially or entirely encoded by the sequence of interest is known (but that need not be the case), and the sequence of interest includes sequences encoding at least one such activity or a portion of such activity.

“Secondary structure” refers to a structure in a nucleic acid molecule that is more than the primary linear structure of the sequence of bases. Secondary structures can include a variety of configurations based at least in part on base-pairings, such as stem-loop configurations or hairpin configurations. (See, U.S. Pat. No. 5,270,163 to Gold et al., issued Dec. 14, 1993; and U.S. Pat. No. 5,747,253 to Ecker, issued May 5, 1998).

“Stem-forming sequence” is a sequence of bases in a nucleic acid molecule that is comprised of two half-stem sequences wherein the first half of the stem-forming sequence is able to base pair with the second half stem-forming sequence when the first and second stem-forming sequences are in single-stranded form. Stem-forming sequences may base-pair to form a double-stranded structure in a continuous strand of nucleic acids. Such double-stranded structures may be of any length, and may be a part of larger secondary structures of the nucleic acid molecule, such as stem-loop structures and hairpin structures, as they are known in the art.

“Substance of interest” refers to a compound that has been selected for screening peptides or complexes of the present invention, or has been identified using the methods of the present invention.

“Solid support” refers to any solid support that can be used in a method of the present invention. Preferably, a solid support is used to immobilize a nucleic acid molecule of the present invention or a complex of the present invention. In addition, a solid support can be used to immobilize a substance of interest, a cell, an etiological agent, or other moiety. Solid substrates can take any form, such as sheets, membranes (such as nitrocellulose or nylon), polymeric surfaces, including wells (such as microtiter wells), beads, or chips, such as glass, nylon, or silica sheets that comprise arrays of nucleic acids, proteins, or other molecules. Solid supports can be of any appropriate material, such as polymers, metals, glass, or silica and can be magnetic in nature. Preferred solid substrates include polystyrene, polycarbonate, latex, polyacrylamide, sepharose, nylon, nitrocellulose, glass, silica, and magnetite.

“On or within a cell” refers to a moiety, such as a receptor or biomolecule that resides on the surface of a cell, within the outer membrane of a cell, or within a cell. Within a cell refers to any locus within a cell, such as in the cytoplasm or within or associated with an organelle, such as, for example, a mitochondria, nucleus or Golgi apparatus.

A “cell” refers to any cell, such as a of prokaryotic (such as bacterial) or eukaryotic origin. Eukaryotic cells include, for example, single cell organisms such as yeast and multicellular organisms such as invertebrates, plants and vertebrates. Invertebrates include parasites such as worms and vertebrates include cold-blooded organisms (such as reptiles and amphibians) and warm-blood organisms, such as mammals, including humans. A cell can be part of a sample of tissue, fluid or organ of a multicellular organism, or can be part of a multicellular organism itself.

“In vitro” refers to procedures that are performed outside of a cell. For example, purified enzymes or extracts of cells can be used to perform procedures in a vessel, such as a test tube.

“Ex vivo” refers to procedures that are performed outside of a multicellular organism, but use whole cells. For example, live cells from a subject, such as a human, can be cultured outside of the body and these cells can be used in testing procedures.

“In vivo” refers to procedures that are performed on a whole organism, such as a subject, including a human, such as in clinical trials. In vivo procedures can also be performed on non-human subjects, such as animal models.

A “normal cell” refers to a cells whose processes and characteristics are in conformance with an average cell of that type. For example, a normal lung cell does not exhibit the proliferation and metastatic capabilities of a cancerous lung cell.

An “abnormal cell” refers to a cell whose processes and characteristics are not in conformance with an average cell of that type. For example, a normal CD4+ does not exhibit the lifespan of a CD4+ cell infected with a virus, such as HIV.

A “neoplastic cell” refers to a cell that exhibits the processes and characteristics of a neoplasm, such as tumors, cancers, carcinomas and the like.

A “virus infected cell” refers to a cell that has been infected with a viable virus and exhibits or will exhibit characteristics of that infection.

An “etiological agent” refers to any etiological agent, such as bacteria, parasites, fungi, viruses, prions and the like.

A “library” refers to a group of two or more compounds or compositions. The members of a library can be mixed into a single population, such as in a single container. Alternatively, the members of a library can be provided separately in different containers, such as in microtiter plates or separate containers in a larger container, such as vials in a box. Alternatively, such separate containers can include one or more members of a library.

Other technical terms used herein have their ordinary meaning in the art that they are used, as exemplified by a variety of technical dictionaries, such as the McGraw-Hill Dictionary of Chemical Terms and the Stedman's Medical Dictionary.

Introduction

As a non-limiting introduction to the breath of the present invention, the present invention includes several general and useful aspects, including:

1) a nucleic acid molecule comprising a transcription regulatory region, an open reading frame comprising a random sequence or sequence of interest, and a transcription termination moiety. The nucleic acid molecule preferably comprises a linking moiety that can link a polypeptide encoded by the open reading frame to the nucleic acid molecule, so that transcription elongation ternary complexes can form. Such nucleic acid molecules can be provided in vectors,

2) a DNA molecule that comprises a linking moiety and an open reading frame comprising a random sequence or sequence of interest, and is directly used as the template for protein translation by ribosome,

3) methods of linking nucleic acid molecules of the present invention to peptides or polypeptides that are encoded by the nucleic acid molecules.

4) libraries of nucleic acid molecules of the present invention, either alone, or linked to peptides or polypeptides they encode. A library of nucleic acid molecule of the present invention can comprise nucleic acid molecules in vectors. A library of nucleic acid molecule of the present invention can be with or without a substance of interest.

5) methods of identifying nucleic acid molecules.

6) methods for identifying peptides or polypeptides.

7) methods of identifying test compounds and test compounds identified by such methods, and pharmaceutical compositions based on identified test compounds.

8) methods of identifying targets and targets, including pharmaceutical targets, identified by such methods.

These aspects of the invention, as well as others described herein, can be achieved using the methods, articles of manufacture and compositions of matter described herein. To gain a full appreciation of the scope of the present invention, it will be further recognized that various aspects of the present invention can be combined to make desirable embodiments of the invention.

Embodiments Using Transcription and Translation Systems

I Nucleic Acid Molecules

The present invention includes nucleic acid molecules that are useful for a variety of purposes, including methods of the present invention. The nucleic acid molecules can be provided in vectors. The nucleic acid molecule can be any nucleic acid molecule that can be transcribed by RNA polymerase, but preferably comprises double-stranded DNA, single-stranded DNA, or partially double-stranded or single-stranded oligonucleotides, but is most preferably DNA that is at least partially double-stranded. A nucleic acid molecule of the present invention preferably comprises a transcription regulatory region, at least one random sequence or sequence of interest, and at least one transcription termination moiety, and preferably comprises or is bound to a linking moiety. In the alternative, a nucleic acid molecule of the present invention can encode a linking moiety that is a region or domain or a polypeptide.

Linking moieties can be any compounds that can directly or indirectly link a polypeptide to a nucleic acid molecule of the present invention that encodes it. Preferably, a linking moiety is covalently bound to a nucleic acid molecule of the present invention. However, this is not a requirement of the present invention.

An example of a linking moiety is puromycin, or another tRNA mimetic, that is bound to a nucleic acid molecule of the present invention can be incorporated into a polypeptide by ribosome. Methods of binding puromycin to the 3′ and 5′ ends of nucleic acid molecules are known in the art, see for example, PCT applications WO 00/72869, WO 01/07657, WO 01/04265, and WO 00/32823, herein incorporated by reference. A linking moeity such as puromycin can also be bound to a DNA binding molecule, such as lac repressor protein or the RNA polymerase itself. In this case, the linkage between a DNA molecule of the present invention that includes a binding site for the DNA binding protein and a polypeptide encoded by the DNA molecule is via the bridge of DNA-DNA binding molecule-puromycin-polypeptide. Preferably puromycin that is bound to a protein (including RNA polymerase) is bound to the protein by a linker (such as a carbon chain) that can allow the puromycin access to a ribosome that translates the RNA transcribed by the RNA polymerase. The puromycin can be incorporated into a polypeptide by a ribosome, and thereby linked, via RNA polymerase, to a DNA molecule that encodes the polypeptide.

It is also possible to synthesize nucleic acid molecules that comprise, at or near a 3′ or 5′ terminus, linking moieties such as, but not limited to, biotin, digoxigenin, nitrilotriacetic acid, nucleic acid sequences, peptide nucleic acid sequences, or peptide sequences. Such nucleic acid molecules can optionally be oligonucleotides that can be used as primers that become incorporated into the 5′ ends of nucleic acid molecules of the present invention in polymerase reactions. Moieties can also be attached to nucleic acid molecules, such as oligomers, that can optionally be hybridized and then ligated to the 3′ ends of nucleic acid molecules of the present invention. In these aspects, a portion of the nascent polypeptide synthesized by the ribosome can be a binding domain that can specifically bind a linking moiety. This region or domain of the polypeptide encoded by a nucleic acid molecule of the present invention can bind to the nucleic acid molecule or to a linking moiety that is directly or indirectly linked to the nucleic acid molecule, such as those listed above. In these embodiments, the nucleic acid construct, in addition to comprising a random sequence or sequence or interest, also comprises a sequence encoding the amino acid sequence of the polypeptide linking domain. Preferably the sequence of interest or random sequence and the sequence encoding the polypeptide linking domain are both part of the same open reading frame.

In some aspects of the invention, a nucleic acid molecule of the present invention does not comprise a linking moiety. In these aspects of the invention, a ribosome can be a linking moiety, and a DNA molecule of the present invention can be linked to the polypeptide it encodes via the following linkages: DNA-RNA polymerase-RNA-ribosome-polypeptide.

The exact nature of these linking moieties, such as the types of compound and sequence and length of nucleic acid or peptide that relate to the function of these structures can be selected based on reports in the literature, or by screening compounds for the desired activity using standard assay methods as they are known in the art. Experiments and assays that test for the linkage of a polypeptide to its own encoding nucleic acid molecule can be designed using, for example, labels that can be incorporated into nucleic acid molecules and polypeptides, and by using separation techniques such as gel electrophoresis and enzymes such as nucleases and proteases to demonstrate the coupling of a nucleic acid molecule to the polypeptide it encodes. See, for example, PCT application number WO 98/31700 and PCT application number WO 00/26511.

The transcription regulatory region can be a prokaryotic promoter that controls the transcription of the DNA to RNA by DNA-dependent RNA polymerase, such as, but not limited to, E. coli, T7, T3, or SP6 RNA polymerase. The transcription regulatory region can also be a eukaryotic promoter and, optionally, enhancer, that controls the transcription of the DNA by an RNA polymerase of a eukaryotic source, for example, RNA polymerase II from HeLa cells. Transcription regulatory regions or “promoters” and “enhancers” are well known in the art, as are assays for determining the promoter and enhancer activity of DNA sequences. Accordingly, transcription regulatory control regions can be those known in the art, modified versions of those known in the art, or later determined.

A random sequence or sequence of interest can comprise either complete random sequence or partially random sequence or semi-random sequence, or a specified sequence combined with a complete random sequence or partially random sequence or semi-random sequence, or can be any sequences, known or unknown, for example of cellular or viral origin. A random sequence or sequence of interest can be of any length, but is preferably from about twelve to about 10,000, more preferably from about twenty to about 5,000, and most preferably from about thirty to about 1,000 bases in length.

A transcription termination moiety is a nucleic acid sequence or structure, a compound covalently or non-covalently attached to the DNA, or a molecule or any entity that binds to the DNA, so that the migration of RNA polymerase along the DNA template is impeded at the transcription termination moiety site. The migration of RNA polymerase can stop, can pause, or can be slowed at the transcription termination site with respect to its migration in the absence of a transcription termination moiety site. Examples of transcription termination moieties are drug molecules (Shi et al, (1988) J. Mol. Biol. 199, 277-293; Shi et al., (1988) J. Biol. Chem. 263, 527-534); White R. J. & Philips, D. R. (1989) Biochemistry 28, 4277-4283); Corda, Y. et al, (1991) Biochemistry 30, 222-230) or site-specific DNA binding proteins (Sancer, G. B. et al, (1982) Cell 28, 523-530; Sellitti, M. A. et al, (1987) Proc. Natl. Acad. Sci. USA 84, 3199-3203; Pavco, P. A. & Steege, D. A., (1990) J. Biol. Chem. 265, 9960-9969; Thomsen, B. et al, (1990) J. Mol. Biol. 215, 237-244; Sastry, S. S. &Hearst, J. E. (1991) J. Mol. Biol. 221, 1091-1110). Nucleic acid sequences or secondary structures that can be used for transcription termination moieties are also known in the art (Arndt and Chamberlin (1990) J. Mol. Biol. 213: 79-108) and can be selected according to published literatures or from nucleic acid libraries using appropriate methods.

The transcription regulatory region, the ORF sequence, the transcription termination moiety and the linking moiety need not be directly linked together, immediately adjacent to each other or be part of the same nucleic acid molecule, but are preferably operably linked. These elements of the nucleic acid molecule of the present invention are preferably provided on a nucleic acid construct in the order of transcription control region, random sequence or sequence of interest, transcription termination region and linking moiety. However, this order can be completely or partially altered in some cases.

The nucleic acid molecules of the present invention can be made using any appropriate method, including synthetic methods or cloning methods as they are known in the art (Sambrook et al., supra, (1989)).

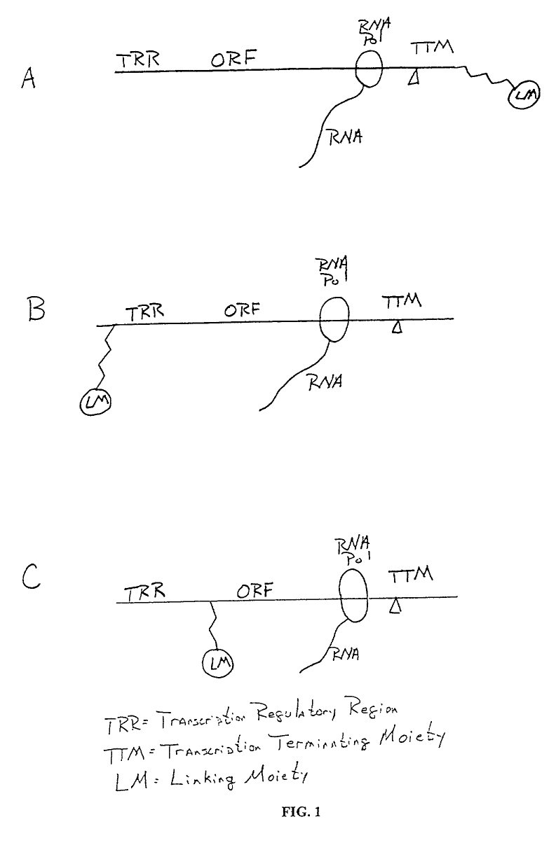

In one aspect of the present invention, exemplified in FIG. 1, the nucleic acid molecule comprises double-stranded DNA and the linking moiety is puromycin that is covalently bound to the DNA. Preferably, the DNA also encodes a ribosome binding RNA sequence that promotes translational intitiation and a translation start codon, such as dAdTdG. In this embodiment of the invention, although the linking moiety is preferably covalently linked to the DNA downstream of the transcription termination moiety (TTM) (a), it also may be linked to the upstream of the transcription termination moiety (TTM) (b), or anywhere along the DNA (c).

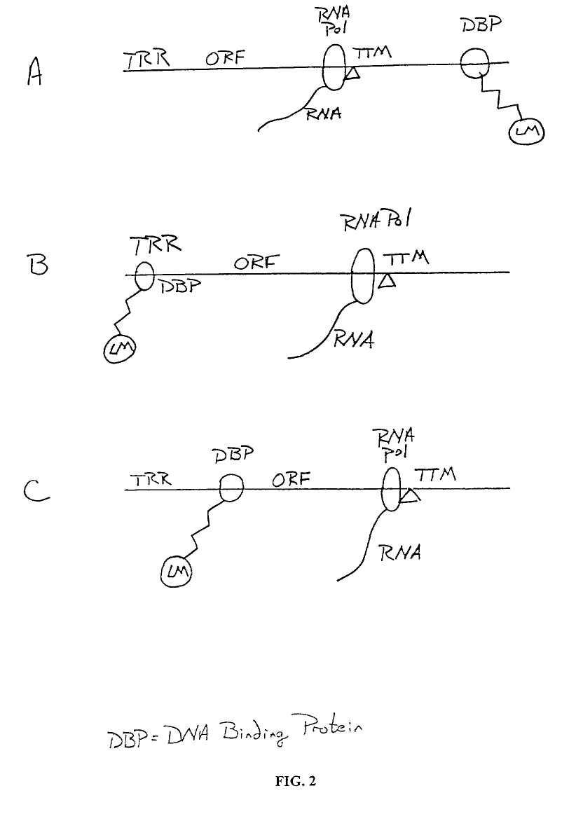

In another aspect, exemplified in FIG. 2, the nucleic acid molecule comprises double-stranded DNA. Preferably, the double-stranded DNA also encodes a ribosome binding RNA sequence and a downstream translation start codon. The DNA also comprises a specific protein binding region that can be recognized and specifically bound by a DNA binding protein (DBP). The protein that specifically binds to the protein binding region of the DNA can be directly or indirectly bound to a linking moiety, such as puromycin. In this embodiment of the invention, the protein binding region of the DNA molecule can be located either downstream of the transcription termination moiety (a) or upstream or the transcription regulatory region (b), or anywhere along the DNA (c).

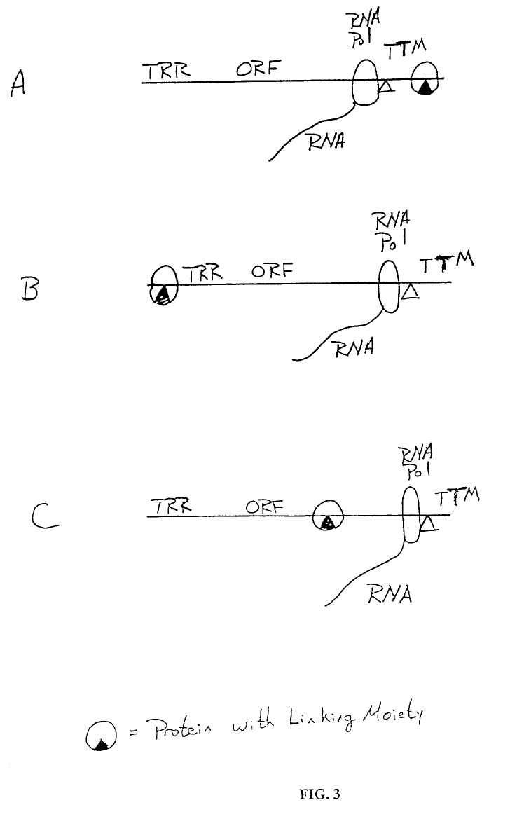

In yet another aspect, exemplified in FIG. 3, the nucleic acid molecule comprises double-stranded DNA that preferably encodes a ribosome binding RNA sequence and a translation start codon. The DNA molecule also comprises a specific protein binding region that can be recognized and specifically bound by a DNA binding protein (DBP). In this embodiment of the invention, the DNA molecule further comprises a sequence that encodes a peptide that can bind to a domain of the protein or compound attached to the DNA binding protein. The protein binding region of the DNA molecule can be located either downstream of the transcription termination moiety (TTM) (a) upstream of the transcription regulatory region (TTM) (b), or anywhere along the DNA (c).

Nucleic acid molecules of the present invention can be of any length in total, but are preferably between about twenty bases and about 10,000 bases, more preferably between about forty bases and about 1,000 bases.

The nucleic acid molecule of the present invention can further include at least one random sequence or at least one sequence of interest or a combination of at least one random sequence and at least one sequence of interest. The random sequence or sequence of interest can be made using appropriate methods in the art, such as by cloning techniques, including PCR techniques and other enzymatic techniques such as, but not limited to, reverse-transcription from cellular mRNA; by solid phase synthesis; by fragmenting nucleic acid molecules using a variety of methods, such as sheer forces, vibrational energy or restriction enzymes; or by any combination of these methods. For the random sequences from synthetic origins, the polynuleotides can be of any length, but are preferably between about twenty bases and about 500 bases, more preferably between about forty bases and about 150 bases in length. For the random sequences from biological origins such as those derived from mRNAs encoding antibodies or families of receptors or ligands, the nucleic acid molecule can be of any length, preferably between about fifty bases and 10,000 bases, more preferably between about 100 bases and 1,000 bases.

Random sequences made by chemical synthesis can be completely random, in which case no bias is given to the incorporation of particular nucleotides (preferably A, C, G, and T) in any position in the synthesized nucleic acid. Alternatively, semi-random sequences can be synthesized by specifying that certain subsets of one or more nucleotides can be employed for one or more positions in the sequence. A random sequence can also be partially random, in which some positions in the sequence are specified, and others are completely random or semi-random.

The linking moiety and the nucleic acid molecule of the present invention can be operably linked. Preferably, the linking moiety and the nucleic acid molecule of the present invention are covalently linked, but this is not a requirement of the present invention. A linking moiety that is bound to a nucleic acid molecule can be 5′ to the sequence encoding the random sequence or sequence of interest or 3′ to the random sequence or sequence of interest. The nucleic acid molecules of the present invention can be made using any appropriate method, including synthetic methods or cloning methods as they are known in the art (Sambrook et al., supra, (1989)).

The nucleic acid molecule of the present invention can further include sequences or other polymers that function as at least one spacer region. A spacer region can include nucleic acid sequences and long chain polymers that preferably do not interact with nucleic acids. A spacer region can be made using appropriate methods in the art, such as solid phase synthesis or cloning methods known in the art. A spacer region can be of any length, but is preferably between about ten bases and about 100 bases, more preferably between about twenty bases and about fifty bases in length, and of equivalent length if the spacer is non-nucleic acid molecule. Preferred spacer regions include secondary structure-free DNA sequences, long chain carbon molecules, or combination of both. A spacer region can located in any portion of a nucleic acid molecule of the present invention. Preferably, though, a spacer region occurs between an open reading frame and a linking moiety.

The transcription control region, ORF, transcription termination region, and linking moiety need not be directly linked together, immediately adjacent to each other or provided on the same nucleic acid molecule, but are preferably operably linked. These elements of the nucleic acid molecule of the present invention can be provided on a nucleic acid construct in any order or orientation. The linking moiety can be anywhere in the nucleic acid, preferably at or near the 5′ or 3′-end of the nucleic acid molecule (see FIGS. 1, 2, and 3). The nucleic acid molecules of the present invention can be made using any appropriate method, including synthetic methods or cloning methods as they are known in the art (Sambrook et al., supra, (1989)) and described in the previously.

The nucleic acid molecules of the present invention may further include sequences that encode peptides that mediate the entry of peptides and other molecules into cells. Such sequences include sequences that encode portions of the tat gene of HIV (Anderson et al. Biochem. Biophys. Res. Commun. 194: 876-884 (1993); Fawell et al., Proc. Natl. Acad. Sci. USA 91: 664-668 (1994); Kim et al., J. Immunol. 159: 1666-1668 (1997); Vives et al. J. Biol. Chem. 272: 16010-16017 (1997); Vocero-Akbani et al. Nat. Med. 5: 29-33 (1999)) and portions of the Drosophila Antennapedia gene (Derossi, et al. J. Biol. Chem. 269: 10444-10450 (1994)) and other sequences that encode peptides that mediate entry of proteins into cells as they are known or become known in the art. Furthermore, the nucleic acids of the present inventions may further include sequences that encode peptides that direct molecules to particular cellular compartments, for example, the endoplasmic reticulum or the mitochondria, as such sequences are known in the art or are later identified or developed.

The nucleic acid molecule of the present invention preferably include at least one control sequence, such as an expression control sequence, that drives or regulates the transcription and/or translation of the nucleic acid molecule of the present invention. For translation, preferred control sequences are “Shine-Dalgarno” sequences or “Kozak sequences” and at least one start codon and with or without a stop codon. However, in some applications, the nucleic acid may not include such a control sequence.

The nucleic acid molecules of the present invention may be directly or indirectly labeled with a detectable marker. The detectable marker may be a radioisotope or a nonradioactive detectable molecule such as biotin or fluorescein, or other detectable markers as they are known or developed in the art. The marker may be directly or indirectly bound to the nucleic acid.

Constructs in Cells

The nucleic acid molecule of the present invention and complexes that include such nucleic acid molecules can also be provided in a cell. The nucleic acid molecule can be introduced into a cell using methods known in the art, such as lipofection or electroporation. In addition, nucleic acid molecules can be introduced into cells using vectors, such as viruses or phages. The cells can be any cell, including prokaryotic or eukaryotic cells, and can be ex vivo or in vivo, including within a whole organism, including a mammal, including a human. Once introduced into a cell, the nucleic acid molecule can be transcribed and/or translated to produce a complex of the present invention.

Constructs in Vectors

The nucleic acid molecules of the present invention, including those that optionally include a sequence of interest or a random sequence, can also be provided in a vector. Vectors can be viral vectors, liposomes, microspheres, plasmids, phages or a linear dsDNA molecules. Vectors preferably include double-stranded DNA molecules of the present invention, but the invention is not limited to such vectors. For example, various viral vectors include double- or single-stranded DNA (parvoviruses), single-stranded RNA (retroviruses) or double-stranded RNA (rotaviruses). Vectors are useful for making libraries of nucleic acid molecules of the present invention, particularly libraries that include nucleic acid molecules that have different random sequences or sequences of interest. Furthermore, such vectors are convenient for making, storing and transporting nucleic acid molecules of the present invention. Vectors can be made or modified using methods in molecular biology as they are known in the art (Sambrook et al., supra, (1989)). The vector of the present invention can be of any vector as that term is known in the art.

Vectors can be, for example, retroviruses (U.S. Pat. No. 5,399,346 to Anderson et al., issued Mar. 21, 1995, Bandara et al., DNA and Cell Biol. 11:227-231 (1992)); adenoviruses (Berkner, BioTechniques 6: 616-629 (1989); adeno-associated viruses (Larrick and Burck, Gene Therapy. Application of Molecular Biology, Elsevier, New York (1991); plasmid vectors (U.S. Pat. No. 5,240,846 to Collins et al., issued August 31, 1993); liposomes (Holmberg et al., J. Liposome Res. 1:393-406 (1990) and Liu et al., Nature Biotechnology 15:167-173 (1997)); microspheres (Mathlowitz et al., Nature 386:410-_(1997)); see generally Larrick et al., Gene Therapy, Elsevier, N.Y. (1991) and Pinkert, Transgenic Animal Technology, Academic Press, San Diego (1994).

The present invention also includes a cell that includes or has been transfected or transformed by a vector of the present invention. Such a cell can be ex vivo or in vivo in a subject, including a test animal or a human. Preferably, the nucleic acid molecule of the present invention in the vector can be expressed in the cell. In one aspect of the present invention, the nucleic acid molecule includes a sequence of interest such that when translated retains at least one activity of the polypeptide encoded by the sequence of interest Alternatively, the vector includes at least one random sequence. The activity of the polypeptide encoded by the random sequence in the cell can be monitored by observing or interrogating the cell using methods known in the art, including the use of reporter genes to report changes in signal transduction within a cell.

Nucleic Acid Molecules with Peptides

The nucleic acid molecule of the present invention can be operably linked to the polypeptide it encodes. Preferably, the operable link between a nucleic acid molecule and a polypeptide of the present invention occurs with covalent bonding, but this is not a requirement of the present invention. After transcription of DNA into RNA, a ribosome can translate the RNA into a polypeptide. Preferably, a linking moiety is covalently bound to DNA and is incorporated into the peptide, providing a nucleic acid molecule bound to the polypeptide it encodes.

Complexes comprising nucleic acid molecules of the present invention operably linked to polypeptides may also be labeled with a detectable label. The detectable label may be a radioisotope or a nonradioactive detectable molecule such as biotin, or other detectable moieties as they are known or developed in the art. The label may be directly or indirectly bound to the nucleic acid of the complex or to a polypeptide of the complex, or both. The polypeptide of the complex labeled with a detectable marker need not be the polypeptide encoded by or partially encoded by the random sequence or sequence of interest.

Construct Bound with a Substance of Interest

In another aspect of the present invention, a nucleic acid molecule of the present invention can be linked to a polypeptide encoded by a random sequence or sequence of interest. The polypeptide can bind with a substance of interest. This aspect of the invention allows the selection of polypeptides that bind with a substance of interest, while at the same time selecting the nucleic acid molecule that encodes the polypeptide that binds with a substance of interest. The substance of interest can be on a solid support, and can be immobilized on such a solid support using methods known in the art such as absorption, chemical conjugation or cross-linking.

Alternatively, the structure formed by a nucleic acid of the present invention and a polypeptide encoded by a random sequence or sequence of interest can be immobilized on a solid support and one or more substances of interest may be bound with or capable of reacting with the fixed complex or complexes.

Furthermore, the substance of interest can be on or within a cell, and the cell can optionally be immobilized on a solid support using appropriate methods, such as solid supports covered with fibronectin or other adhesion molecules, entrapped thereon. The cell can be ex vivo and can be provided as a cell, culture of cells, or part of a sample of tissue, fluid or organ. Alternatively, the cell can be in vivo in a subject.

The substance of interest may be a cell-type-specific or tissue-specific molecule. Nucleic acids or peptides of the present invention that specifically bind to the substance of interest can be identified. Peptides that bind such cell-type-specific and tissue-specific molecules can be used to target drug delivery to specific cells or tissues.

The cell can be any cell, including a normal cell or an abnormal cell, such as, for example, a neoplastic cell or a virus infected cell. The substance of interest can also be on or within an etiological agent, such as, for example, a virus, a bacteria, a bacterial spore, a parasite or a prion. The substance of interest may be one or a plurality of molecules on or within an etiological agent, virus, bacterium, protozoan, tumor cell or abnormal cell. The substance of interest used for selection may be whole cells, viruses, or microorganisms fixed to a solid support or in solution, or may be a portion or fractionated preparation of one or more cells, viruses, or microorganisms fixed to a solid support or in solution.

The substance of interest can also include at least one organic molecule, an inorganic molecule, a polymer, a polypeptide, a lipid, a carbohydrate, a small molecule, a nucleic acid molecule, a ribozyme, a biomacromolecule or a drug.

II Libraries

The present invention also includes a library of nucleic acid molecules of the present invention. Such libraries include nucleic acid molecules that contain linking moieties so that they are able to covalently or noncovalently bind to the polypeptides they encode.

The library of nucleic acid molecules can include at least two different random sequences, at least two different sequences of interest or a combination of at least one random sequence and at least one sequence of interest. For example, a library of nucleic acid molecules can include two or more such nucleic acid molecules. Each nucleic acid molecule can have a different random sequence or a different sequence of interest. In addition, one or more nucleic acid molecule can have a random sequence and the other nucleic acid molecule can have a sequence of interest.

The library can optionally be fixed to a solid support. In particular, libraries containing random sequences, which may include sequences in which one or a few sequence positions have been randomly or semi-randomly varied, or libraries containing sequences of interest, can be fixed to a chip or array for screening with one or more substances of interest. A translated library of complexes can also be fixed to a solid support. In particular, libraries of complexes containing random sequences, which may include sequences in which one or a few sequence positions have been randomly or semi-randomly varied, or libraries of complexes containing sequences of interest, can be fixed to a chip or array for screening with one or more substances of interest.

Members of the libraries of the present invention may be labeled with a detectable label. The detectable label may be a radioisotope or a nonradioactive detectable molecule such as biotin, or other detectable moieties as they are known or developed in the art. The label may be directly or indirectly bound to the members of the library. Library members may be labeled by direct or indirect binding of a detectable marker to the nucleic acid or to a polypeptide of the library member.

Library with a Substance of Interest

A library of nucleic acid molecules can also include at least one substance of interest. The substance of interest can be bound with a nucleic acid molecule, a polypeptide, or complex of the present invention, can be unbound, or can be bound to some members of the library and not bound to other members of the library. The substance of interest can be directly bound or indirectly bound to a nucleic acid molecule of the present invention, but is preferably indirectly bound to a nucleic acid molecule of the present invention or directly bound to a complex of the present invention (particularly the polypeptide encoded by the random sequence or sequence of interest). Alternatively, the substance of interest can be a substrate, such as an enzymatic substrate, with which a nucleic acid molecule, polypeptide, or complex of the present invention interacts. Reactions of the nucleic acid molecule, polypeptide, or complex of the present invention with the substance of interest may be monitored and quantitated using appropriate assays, for example spectrophotometric assays or assays that measure the release of a radioactive moiety. The substance of interest can be directly or indirectly bound on a solid support or in solution.

In an alternative format, the structure formed by the construct of the present invention and the polypeptide encoded by a random sequence or sequence of interest can be immobilized on a solid support and one or more substances of interest may be unbound, may be bound with the fixed one or more complexes of the library, or may be acted upon in a biochemical reaction catalyzed by or modulated by one or more complexes of the library.

The substance of interest can be on or within a cell, wherein the cell can be ex vivo or in vivo, such as in a subject. The cell can be any cell, including a normal cell or an abnormal cell, such as, for example, a neoplastic cell or a virus infected cell. The substance of interest can also be one or a plurality of molecules on or within an abnormal or normal cell or on or within an etiological agent, such as, for example, a virus, a bacterium, a bacterial spore, a parasite or a prion. The substance of interest may be one or a plurality of molecules on or within an etiological agent, virus, bacterium, protozoan, tumor cell or abnormal cell. The substance of interest used for selection may be whole cells, viruses, or microorganisms fixed to a solid support or in solution, or may be a portion or fractionated preparation of one or more cells, viruses, or microorganisms fixed to a solid support or in solution.

The substance of interest may be a cell-type-specific or tissue-specific molecule, such that nucleic acids or peptides of the present invention that specifically bind to the substance of interest can be identified. Such cell-type-specific and tissue-specific molecules can be used to target drug delivery to specific cells or tissues.

The substance of interest can also include at least one organic molecule, an inorganic molecule, a polymer, a polypeptide, a lipid, a carbohydrate, a small molecule, a nucleic acid molecule, a ribozyme, a biomacromolecule or a drug.

Library of Vectors

The present invention also includes a library of vectors of the present invention. Preferably, the library of vectors includes at least two different random sequences, at least two different sequences of interest, or a combination of one or more random sequences and one or more sequences of interest.

III Methods for Identifying Nucleic Acid Molecules

The present invention includes methods for identifying nucleic acid molecules, particularly from a library of random sequences or sequences of interest that encode polypeptides that can bind with a substance of interest.

This method includes: providing at least one nucleic acid molecule of the present invention that comprises at least one open reading frame, where the open reading frame comprises at least one random sequence or at least one sequence of interest; transcribing the nucleic acid molecule to form at least one transcription complex, wherein the transcription complex comprises a nucleic acid molecule operably linked to the its own transcribed RNA; translating the RNA to form a nucleic acid-polypeptide complex, wherein the nucleic acid peptide complex comprises a nucleic acid operably linked to a polypeptide that the nucleic acid encodes; contacting at least one nucleic acid-polypeptide complex with at least one substance of interest; selecting at least one complex that binds with the at least one substance of interest; and identifying the nucleic acid molecule that comprises at least one random sequence or sequence of interest. The nucleic acid molecule, including the random sequence or sequence of interest can be sequenced, or can be detected by hybridization with probe nucleic acid molecules. Optionally, a nucleic acid molecule can be amplified before it is identified. Optionally, a nucleic acid molecule can be cloned before it is identified.

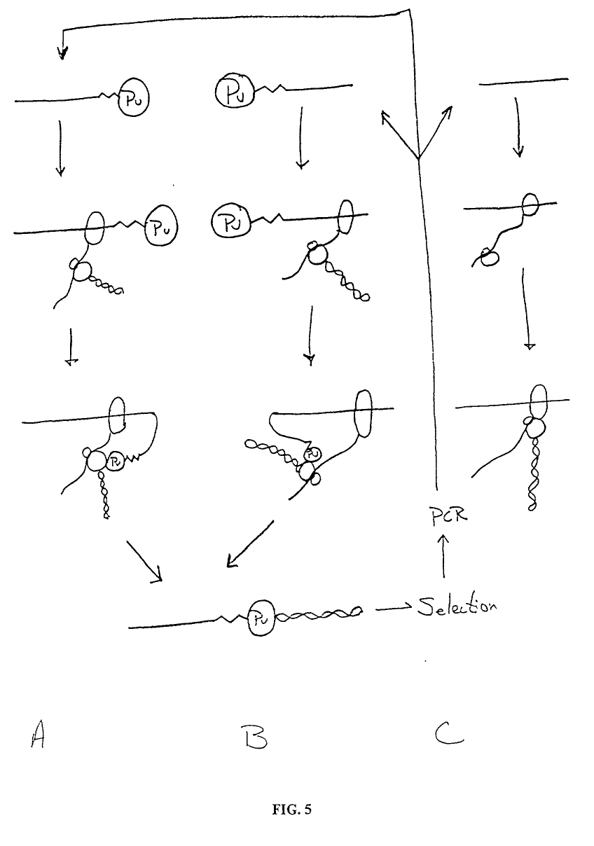

For example, as illustrated in FIG. 5, a DNA molecule of the present invention or a library thereof can be transcribed to RNA by a DNA-dependent RNA polymerase. The transcription reaction is arrested by a transcription termination moiety on the template so that the RNA, RNA polymerase and DNA form a ternary complex. One or more ribosomes can translate the RNA into a peptide or polypeptide. The linking moiety, such as puromycin, which can be attached to either the 3′-(a) or 5′-(b) end of the DNA molecule, can therefore be linked to the nascent polypeptide. The transcription ternary complex is dissociated and the DNA-polypeptide hybrid molecule is released. Both prokaryotic and eukaryotic in vitro transcription and translation systems are well known in the art. It is also possible to perform transcription and translation in vivo, by introducing nucleic acid molecules of the present invention into cells, but this is not preferred. Transcription and translation systems can be used that are compatible with the transcription and translation regulatory sequences of the nucleic acid molecule used in the methods of the present invention. Transcription and translation reactions can be coupled, occurring simultaneously, or can be performed sequentially.

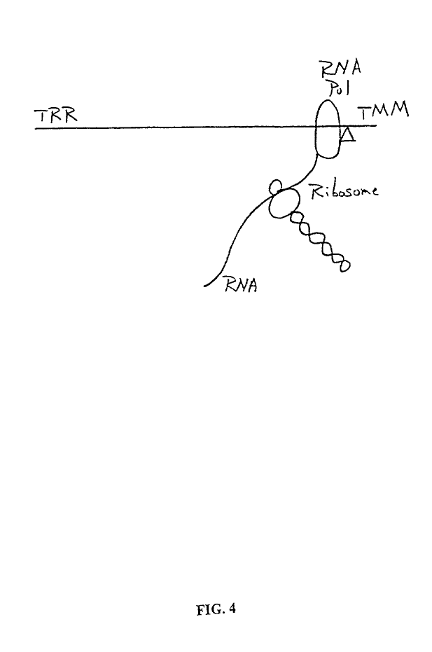

The DNA-polypeptide hybrid is added to a mixture of one or more target compounds and the polypeptide portion of the complex can bind with a target molecule. Bound complexes are separated from the unbound complexes. The nucleic acid molecules on the bound complexes can be eluted from the complex or be used directly as the template for nucleic amplification reactions such as PCR. The amplified nucleic acid can be sequenced or expressed in living organisms. If one of the primers used in amplification reactions is 5′-puromycin linked, the amplified DNA can be used as the template for another round of selection by the same procedures as described above.

Where the linking moiety is other than puromycin or puromycin-like molecules as illustrated in FIGS. 2 and 3, or a ribosome as in FIG. 4, the same basic procedures can also be applied.

It may be desirable in some embodiments, following translation, to incubate the translation mixture under particular conditions of salt or temperature or a time period, and/or with enzymes or chemicals that may enhance the formation or stability of the complexes or may modify the complexes to enhance their efficiency in screening protocols or other applications. The complex may optionally be depleted of ribosomes by treating the mixture of translated constructs with reagents that are known to cause the dissociation of ribosomes from RNA. For example, following translation, EDTA may be added to deplete free Mg²⁺ in the reaction mixture. This may be desirable for screening applications where the ribosome may impede binding to the substance of interest, or impede the entry of complexes into cells.

Complexes, including libraries of complexes, can be purified or substantially purified from a translation reaction mixture using reagents that bind parts of the complex. For example, translated polypeptides can contain a stretched of adjacent six histidine residues so that the DNA-polypeptide complex may be purified from the translation reaction using nitrilotriacetic-linked beads. Such histidines can be encoded by nucleic acid molecules used in the methods of the present invention. Purified or substantially purified complexes may be stored under conditions that promote the stability of nucleic acids and polypeptides, for example, at 4° C. in a buffer that contains BSA and EDTA.

The complex can be contacted with one or more substances of interest under conditions that promote the binding of the complex, or reaction of the complex, particularly the polypeptide encoded by the random sequence or sequence of interest, with the substance of interest. The substance of interest can be on a solid support or in solution. A solid support may be a chip or array. The substance of interest can be on or within a cell and can be on or within an etiological agent. Thus, a substance of interest can bind with a complex that includes a polypeptide encoded by a random sequence or a sequence of interest and the random sequence or sequence of interest itself. Complexes that are not bound to a substance of interest can be separated from bound complexes using methods known in the art. For example, if the substance of interest is bound on a solid support and complexes are bound to the substance of interest or free in solution, the complexes that are free in solution can be washed away using methods known in the art for receptor-ligand reactions, such as immunoassay methods. Alternatively, the complexes of the present invention may be fixed to a chip or array, and the substance or substances of interest may be contacted with the chip or array to allow the substance of interest to bind or react with complexes for which the substance of interest has affinity. The substance of interest may be labeled with a detectable marker, or may be detected with a reagent specific for the substance of interest. Nonspecifically bound substance of interest may be washed off using appropriate methods as they are known in the art prior to detection of the bound substance of interest. Thus, the nucleic acid molecule encoding a peptide that binds with or reacts with a substance of interest has been selected using this method.

The selected complex or portions thereof, such as the polypeptide or the nucleic acid molecule encoding the polypeptide, can be isolated by recovery using a variety of methods. For example, changes in pH, detergents, denaturing agents (such as phenol, urea or guanidinium), concentration and types of salts, such as chaotropic or anti-chaotropic salts, or combinations thereof can be used to elute the complex or portions thereof. Alternatively, the complex can be digested using enzymes, such as proteases or nucleases to free portions of the complex such as the polypeptide or the nucleic acid molecule.

The bound nucleic acid is recovered and enriched using appropriate nucleic amplification reactions, such as polymerase chain reaction (PCR). The enriched nucleic acid can be sequenced using appropriate methods known to the art.

The recovered nucleic acid molecules that contain a random sequence or sequence of interest can also optionally be cloned into appropriate vectors, such as plasmids, which can be amplified in an appropriate host. The recovered nucleic acid, which may contain several DNA species, may also be separated using the methods that exploit the sequence and conformation of the nucleic acid. It may be necessary to separate the double-stranded PCR product to single-stranded in order to use the said method. These methods can be capillary affinity gel for nucleic acid and HPLC, or the combination thereof. The individual species of the nucleic acid molecules can then be sequenced. The amino acid sequence of the polypeptide that is able to bind to the substance of interest can be deduced from the nucleic acid sequence.

The entire selection procedures, or portions thereof, may be automated. Translated complexes can be contacted with targets and unbound complexes can be washed away by a programmable machine. Another component of the automated machine may perform amplification reactions on the nucleic acid molecules of the bound complexes. Several rounds of selection and amplification may be automated in a linked process, and the final PCR products can be separated on a column that utilizing the difference in sequence and conformation. Individual nucleic acid molecules may be transferred directly to an automated sequencer and sequenced, for example using fluorescently tagged nucleotides that may be read spectrophotometrically.

The present invention includes nucleic acid molecules that comprise at least a portion of a random sequence or selected nucleic acid sequence identified by this method. The present invention also includes polypeptides that include at least a portion of a polypeptide encoded by an identified random sequence or sequence of interest.

Other Forms of Nucleic Acid Molecules

The present invention also includes a method for identifying a nucleic acid molecule or sequence in other forms, which include double-stranded DNA, single- or double-stranded RNA or RNA-DNA duplex. The sources for these forms of nucleic acid can be either synthetic or biological such as viral, prokaryotic and eukaryotic. All these nucleic acid must be converted to DNA as the template for the synthesis of RNA that is in turn used as the template for protein translation.

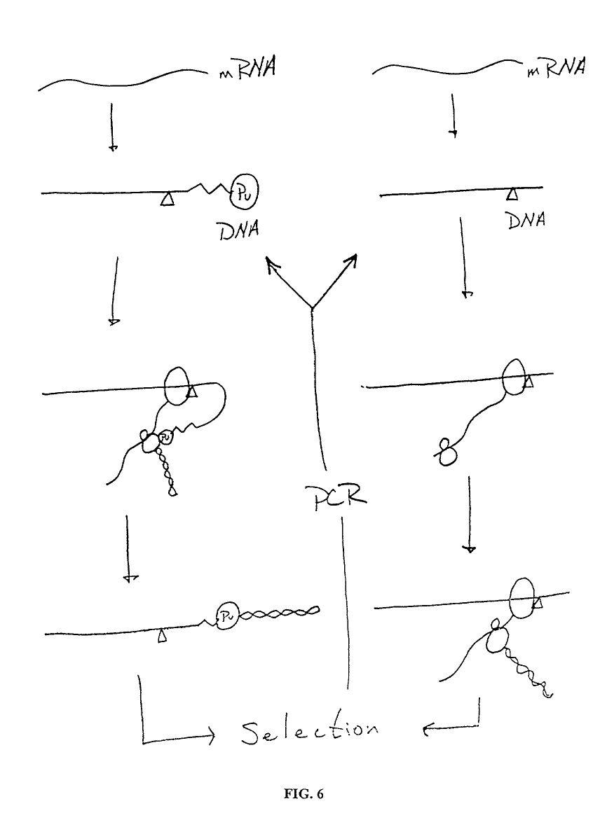

For example (FIG. 6), total eukaryotic cellular mRNA may be converted to DNA using the methods known to the art. The DNA is linked with a linking moiety. Then, the selection and enrichment of the nucleic acid sequences can be carried out using the procedures described in the previous section.

The present invention includes nucleic acid molecules that include at least a portion of a random sequence or selected nucleic acid sequence identified by this method. The present invention also includes polypeptides that include at least a portion of a polypeptide encoded by an identified random sequence or sequence of interest.

IV Methods for Identifying Polypeptides

The present invention includes methods for identifying polypeptides, particularly polypeptides encoded by random sequences or sequences of interest that bind with a substance of interest.

This method includes: providing at least one nucleic acid molecule of the present invention that comprises at least one open reading frame, where the open reading frame comprises at least one random sequence or at least one sequence of interest; transcribing the nucleic acid molecule to form at least one transcription complex, wherein the transcription complex comprises a nucleic acid molecule operably linked to the its own transcribed RNA; translating the RNA to form a nucleic acid-polypeptide complex, wherein the nucleic acid peptide complex comprises a nucleic acid operably linked to a polpeptide that the nucleic acid encodes; contacting at least one nucleic acid-polypeptide complex with at least one substance of interest; selecting at least one complex that binds with said at least one substance of interest; and identifying said random sequence or said DNA sequence of interest. The nucleic acid molecule, including the random sequence, sequence of interest, or selected sequence, can be sequenced. The amino acid sequences of polypeptides that bind to the target molecules can be deduced from the DNA sequence. Optionally, the polypeptides can be synthesized chemically or expressed in living organisms.

For example, as illustrated in FIG. 5, a DNA molecule of the present invention or a library thereof can be transcribed to RNA by a DNA-dependent RNA polymerase. The transcription reaction is arrested by a transcription termination moiety on the template so that the RNA, RNA polymerase and DNA form a ternary complex in which the RNA can be translated to protein. A linking moiety, such as puromycin, which can be attached to either 3′-(a) or 5′-end (b) of the DNA molecule, can therefore be linked to the nascent polypeptide. The transcription ternary complex is dissociated and the DNA-polypeptide hybrid molecule is released.

The DNA-polypeptide hybrid is put in the mixture of target molecules or molecule of interest and the polypeptide portion of the complex binds with the target molecule or the molecule of interest. The bound complex is separated from the bound complexes. The nucleic acid on the bound complex can be eluted from the complex or be used directly as the template for nucleic amplification reactions such as PCR. The amplified nucleic acid can be sequenced or expressed in living organisms. If one of the primer is a 5′-puromycin linked, the amplified DNA can be used as the template for another round of selection by the same procedures as described above.

Where the linking moiety is other than puromycin or puromycin-like molecules as illustrated in FIGS. 2 and 3, or the ribosome in FIG. 4, the same procedures can also be applied.

It may be desirable, following translation, to incubate the translation mixture under particular conditions of salt or temperature or a time period, and/or with enzymes or chemicals that may enhance the formation or stability of the complexes or may modify the complexes to enhance their efficiency in screening protocols or other applications. The complex may optionally be depleted of ribosomes by treating the mixture of translated constructs with reagents that are known to cause the dissociation of ribosomes from RNA. For example, following translation, EDTA may be added to deplete free Mg²⁺ in the reaction mixture. This may be desirable for screening applications where the ribosome may impede binding to the substance of interest, or impede the entry of complexes into cells.

Complexes, including libraries of complexes, may be purified or substantially purified from the reaction mixture using reagents that bind parts of the complex. For example, if the polypeptide contains a stretched of adjacent six histidine residues, the DNA-polypeptide complex may be purified from the translation reaction using nitrilotriacetic-linked beads. Purified or substantially purified complexes may be stored under conditions that promote the stability of nucleic acids and polypeptides, for example, at 4° C. in a buffer that contains BSA and EDTA.

The complex can be contacted with one or more substances of interest under conditions that promote the binding of the complex, or reaction of the complex, particularly the polypeptide encoded by the random sequence or sequence of interest, with the substance of interest. The substance of interest can be on a solid support or in solution. A solid support may be a chip or array. The substance of interest can be on or within a cell and can be on or within an etiological agent. Thus, a substance of interest is bound with a complex that includes a polypeptide encoded by a random sequence or a sequence of interest and the random sequence or sequence of interest itself. Complexes that are not bound to a substance of interest can be separated from bound complexes using methods known in the art. For example, if the substance of interest is bound on a solid support and complexes are bound to the substance of interest or free in solution, the complexes that are free in solution can be washed away using methods known in the art for receptor-ligand reactions, such as immunoassay methods. Alternatively, the complexes of the present invention may be fixed to a chip or array, and the substance or substances of interest may be contacted with the chip or array to allow the substance of interest to bind or react with complexes for which the substance of interest has affinity. The substance of interest may be labeled with a detectable marker, or may be detected with a reagent specific for the substance of interest. Nonspecifically bound substance of interest may be washed off using appropriate methods as they are known in the art prior to detection of the bound substance of interest. Thus, the nucleic acid molecule encoding a peptide that binds with or reacts with a substance of interest has been selected using this method.

The selected complex or portions thereof, such as the polypeptide or the nucleic acid molecule encoding the polypeptide, can be isolated by recovery using a variety of methods. For example, changes in pH, detergents, denaturing agents (such as phenol, urea or guanidinium), concentration and types of salts, such as chaotropic or anti-chaotropic salts, or combinations thereof can be used to elute the complex or portions thereof. Alternatively, the complex can be digested using enzymes, such as proteases or nucleases to free portions of the complex such as the polypeptide or the nucleic acid molecule.

The bound nucleic acid is recovered and enriched using appropriate nucleic amplification reactions, such as polymerase chain reaction (PCR). The enriched nucleic acid can be sequenced using appropriate methods known to the art.

The recovered nucleic acid molecules that contain a random sequence or sequence of interest can be cloned into appropriate vectors, such as plasmids, which can be amplified in an appropriate host. The recovered nucleic acid, which may contain several DNA species, may also be separated using the methods that exploit the sequence and conformation of the nucleic acid. It may be necessary to separate the double-stranded PCR product to single-stranded in order to use such methods. These methods can be capillary affinity gel for nucleic acid and HPLC, or the combination thereof. The individual species of the nucleic acid molecules can then be sequenced. The amino acid sequences of polypeptides that bind to the target molecules can be deduced from the DNA sequence. If desired, the selected polypeptides can be either synthesized chemically or expressed in living organisms.

The entire selection procedures, or portions thereof, may be automated. Translated complexes can be contacted with targets and unbound complexes can be washed away by a programmable machine. Another component of the automated machine may perform amplification reactions on the nucleic acid molecules of the bound complexes. Several rounds of selection and amplification may be automated in a linked process, and the final PCR products can be separated on a column that utilizing the difference in sequence and conformation. Individual nucleic acid molecules may be transferred directly to an automated sequencer and sequenced, for example using fluorescently tagged nucleotides that may be read spectrophotometrically.

The present invention includes nucleic acid molecules that comprise at least a portion of a random sequence or selected nucleic acid sequence identified by this method. The present invention also includes polypeptides that include at least a portion of a polypeptide encoded by an identified random sequence or sequence of interest.

Other Forms of Nucleic Acid Molecules

The present invention also includes a method for identifying a nucleic acid molecule or sequence in other forms, which include double-stranded DNA, single- or double-stranded RNA or RNA-DNA duplex. The sources for these forms of nucleic acid can be either synthetic or biological such as viral, prokaryotic and eukaryotic. All these nucleic acid must be converted to DNA as the template for the synthesis of RNA that is in turn used as the template for protein translation.

For example (FIG. 6), total eukaryotic cellular mRNA may be converted to DNA using the methods known to the art. The DNA is then tagged with a linking moiety. Then, the selection and enrichment of the nucleic acid sequences can be carried out using the procedures described in the previous section.

The present invention includes nucleic acid molecules that include at least a portion of a random sequence or selected nucleic acid sequence identified by this method. The present invention also includes polypeptides that include at least a portion of a polypeptide encoded by an identified random sequence or sequence of interest.

Embodiments Using Translation Systems

V Nucleic Acid Molecules

The present invention includes nucleic acid constructs that are useful for a variety of purposes, including methods of the present invention. A nucleic acid molecule of the present invention preferably comprises a linking moiety and an open reading frame (ORF). Linking moieties are domains of a nucleic acid molecule, or chemical compounds intrinsic to or bound to nucleic acid molecules, that can link with a polypeptide encoded by the ORF. Preferably, the linking moiety is covalently bound to the nucleic acid. However, this is not a requirement of the invention.

Linking moieties can be any compounds that can directly or indirectly link a polypeptide to a nucleic acid molecule of the present invention that encodes it. For example, puromycin, or other tRNA mimetics or amino acid analogs, bound to a nucleic acid molecule of the present invention can be incorporated into a polypeptide catalyzed by ribosome. Methods of binding puromycin to the 3′ and 5′ ends of nucleic acid molecules are known in the art, see for example, PCT applications WO 00/72869, WO 01/07657, WO 01/04265, and WO 00/32823, all herein incorporated by reference. A linking moeity such as puromycin can also be bound to a DNA binding molecule, such as lac repressor protein or the RNA polymerase itself. In this case, the linkage between a DNA molecule of the present invention that includes a binding site for the DNA binding protein and a polypeptide encoded by the DNA molecule is via the bridge of DNA-DNA binding molecule-puromycin-polypeptide. Preferably puromycin that is bound to a protein (including RNA polymerase) is bound to the protein by a linker (such as a carbon chain) that can allow the puromycin access to a ribosome that translates the RNA transcribed by the RNA polymerase. The puromycin can be incorporated into a polypeptide by a ribosome, and thereby linked, via RNA polymerase, to a DNA molecule that encodes the polypeptide.

In some aspects of the present invention, a portion of the nascent polypeptide synthesized by the ribosome can be a linking moiety, and can bind to the nucleic acid molecule or to a compound or any other entity that is directly or indirectly linked to the nucleic acid molecule. In these aspects of the invention, nucleic acid molecules that can comprise, at or near a 3′ or 5′ terminus, linking moieties such as, but not limited to, biotin, digoxigenin, nitrilotriacetic acid, nucleic acid sequences, peptide nucleic acid sequences, or peptide sequences. All of these compounds can be bound by binding domains encoded by the ORF of the nucleic acid molecule. For example, the binding domain can comprise at least a portion of an avidin or streptavidin protein, a sequence of consecutive histidine residues that can bind nitrilotriacetic acid, or at least a portion of an antibody or other specific binding member that binds a linking moiety such as digoxygenin or a peptide coupled to the nucleic acid molecule. Means of attaching such moieties to nucleic acid molecules are known in the art. Oligodeoxynucleotides with attached linking moieties can optionally be used as primers that become incorporated into the 5′ ends of nucleic acid molecules of the present invention in polymerase reactions. Moieties can also be attached to nucleic acid molecules, such as oligomers, that can optionally be hybridized and then ligated to the 3′ ends of nucleic acid molecules of the present invention. In these embodiments, the nucleic acid construct, in addition to comprising a random sequence or sequence or interest, also comprises a sequence encoding the amino acid sequence of the binding domain. Preferably the sequence of interest or random sequence and the sequence encoding the polypeptide binding domain are both part of the same open reading frame.

The exact nature of these linking moieties, such as the types of compound and sequence and length of nucleic acid or peptide that relate to the function of these structures can be selected based on reports in the literature, or by screening compounds for the desired activity using standard assay methods as they are known in the art. Experiments and assays that test for the linkage of a polypeptide to its own encoding nucleic acid molecule can be designed using, for example, labels that can be incorporated into nucleic acid molecules and polypeptides, and by using separation techniques such as gel electrophoresis and enzymes such as nucleases and proteases to demonstrate the coupling of a nucleic acid molecule to the polypeptide it encodes. See, for example, PCT application number WO 98/31700 and PCT application number WO 00/26511, both herein incorporated by reference.

The ORF of the nucleic acid molecule of the present invention can include at least one random sequence or at least one sequence of interest or a combination of at least one random sequence and at least one sequence of interest. The random sequence or sequence of interest can be made using appropriate methods in the art, such as cloning techniques, including PCR techniques and other enzymatic techniques such as reverse-transcription from cellular mRNA, solid phase synthesis, or fragmenting nucleic acid molecules using a variety of methods, such as sheer forces, vibrational energy or restriction enzymes or a combination of these methods. For the random sequences from synthetic origins, the polynucleotides can be of any length, but are preferably between about twenty bases and about 500 bases, more preferably between about forty bases and about 150 bases in length. For the random sequences from biological origins such as those derived from mRNAs encoding antibodies or families of receptors or ligands, the nucleic acid can be of any length, preferably between about 100 bases and 10,000 bases, more preferably between about eighty bases and 1,000 bases.

A sequence of interest can be any sequence of interest, and can be known or unknown, for example, it can be known sequences of one or more proteins, or it can be one or more sequences of an unfractionated, fractionated, or partially fractionated population of nucleic acid molecules. The ORF can also comprises random sequences combined with sequences of interest in any way.

The nucleic acid molecule can be any nucleic acid molecule, but is preferably single-stranded DNA or double-stranded DNA, and can also be a DNA/RNA duplex molecule. The nucleic acid constructs can be provided in vectors with linking moieties. Linking moieties can be chemical groups or compounds that are bound to or preferably incorporated into a nucleic acid molecule and can bind or be incorporated into the encoded polypeptide by the ribosome. For example, tRNA analogues such as, but not limited to, puromycin, incorporated into the nucleic acid can be linked to a polypeptide by ribosome. Puromycin can also be bound to a DNA binding molecule, such as lac repressor protein. In this case, the linkage between a DNA molecule of the present invention that includes a binding site for the DNA binding protein and a polypeptide encoded by the DNA molecule is via the bridge of DNA-DNA binding molecule-puromycin-polypeptide. Preferably puromycin that is bound to a protein (including RNA polymerase) is bound to the protein by a linker (such as a carbon chain) that can allow the puromycin access to a ribosome that translates the DNA. The puromycin can be incorporated into a polypeptide by a ribosome, and thereby linked to a DNA molecule that encodes the polypeptide.

The linking moiety and the ORF sequence encoding a sequence of interest or random sequence need not be directly linked together or immediately adjacent to each other, but are preferably operably linked. These elements of the nucleic acid molecule of the present invention can be provided on a nucleic acid construct in any order or orientation. The nucleic acid molecules of the present invention can be made using any appropriate method, including synthetic methods or cloning methods as they are known in the art (Sambrook et al., supra, (1989)).

Nucleic acid molecules of the present invention can be of any length in total, but are preferably between about twenty bases and about 10,000 bases, more preferably between about forty bases and about 1,000 bases.

The linking moiety and the nucleic acid molecule are operably linked, preferably covalently linked together. The linking moiety can be 5′ to the sequence encoding the interacting domain or 3′ to the random sequence or sequence of interest. The nucleic acid molecules of the present invention can be made using any appropriate method, including synthetic methods or cloning methods as they are known in the art (Sambrook et al., supra, (1989)).

The nucleic acid molecule of the present invention preferably include at least one ribosome binding sequence (RBS), that promotes the initiation of translation of a nucleic acid molecule of the present invention. A ribosome binding sequence, or translation initiation sequence can be, for example, a Shine-Dalgarno sequence, a Kozak sequence, or an IRES sequence. For translation, preferred sequences for the initiation of translation are “Shine-Dalgarno” sequences and at least one start codon. A preferred start codon is dAdTdG. However, in some applications, the nucleic acid may not include such control sequences.

Preferably, a nucleic acid molecule of the present invention also includes a ribosome stalling sequence, where translation is halted or dramatically slowed while the ribosome remains bound to the nucleic acid template. Preferably, a ribosome stalling sequence is positioned 3′ of an open reading frame of a nucleic acid molecule of the present invention. Preferred ribosome stalling sequences for nucleic acid molecules of the present invention include polydA and sequences having stable secondary structure, such as hairpin structure.

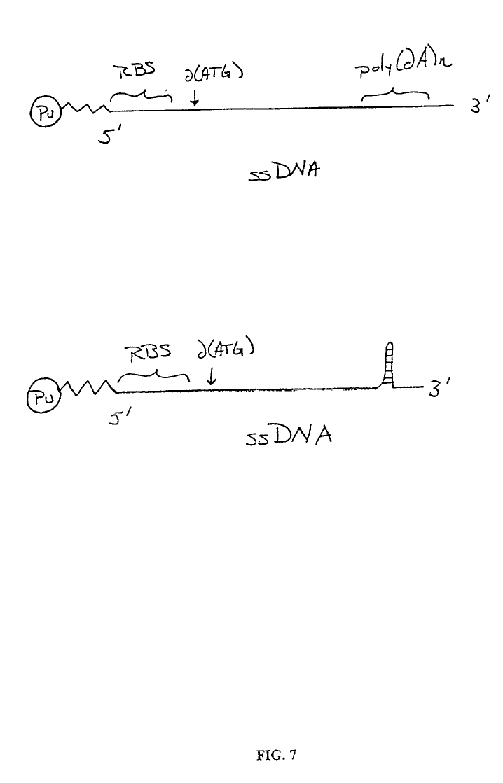

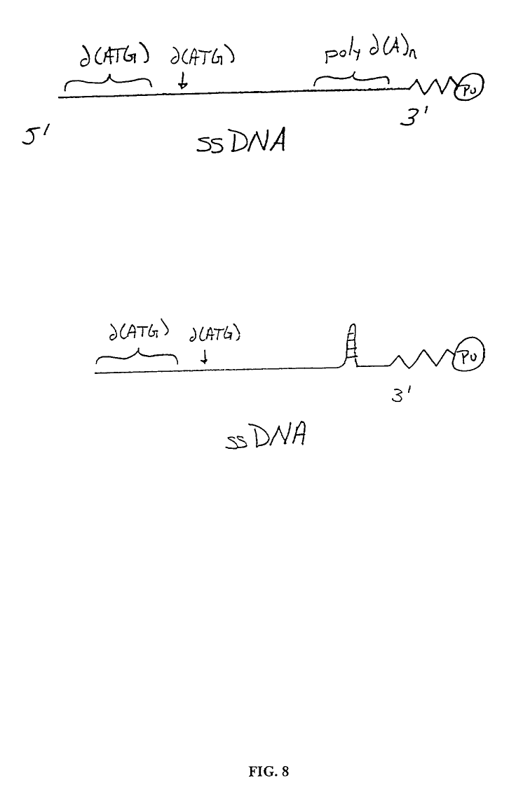

The nucleic acid molecule of the present invention can further include sequences or other polymers that function as at least one spacer region. The spacer region includes nucleic acid sequences or long chain polymers that preferably do not interact with nucleic acids. The spacer region can be made using appropriate methods in the art, such as solid phase synthesis or cloning methods known in the art. The spacer region can be of any length, but is preferably between about five bases and about 100 bases, more preferably between about ten bases and about fifty bases in length, equivalent in length if the spacer is non-nucleic acid molecule. Preferred spacer regions include secondary structure-free DNA sequences, long chain carbon molecules, or combination of both (see FIGS. 7 and 8). Preferably, a spacer region separates a linking moiety from the attached nucleic acid molecule.

The linking moiety, the ribosome binding sequence, sequence encoding polypeptide or the random sequence or sequence of interest, ribosome stalling sequence and spacer region need not be directly linked together, immediately adjacent to each other or provided on the same nucleic acid molecule, but are preferably operably linked. These elements of the nucleic acid molecule of the present invention can be provided on a nucleic acid construct in any order or orientation. The linking moiety can be anywhere in the nucleic acid, preferably at the 5′ or 3′-end of the nucleic acid (see FIGS. 7 and 8). The nucleic acid molecules of the present invention can be made using any appropriate method, including synthetic methods or cloning methods as they are known in the art (Sambrook et al., supra, (1989)).

The nucleic acid molecules of the present invention may further include sequences that encode peptides that mediate the entry of peptides and other molecules into cells. Such sequences include sequences that encode portions of the tat gene of HIV (Anderson et al. Biochem. Biophys. Res. Commun. 194: 876-884 (1993); Fawell et al., Proc. Natl. Acad. Sci. USA 91: 664-668 (1994); Kim et al., J. Immunol. 159: 1666-1668 (1997); Vives et al. J. Biol. Chem. 272: 16010-16017 (1997); Vocero-Akbani et al. Nat. Med. 5: 29-33 (1999)) and portions of the Drosophila Antennapedia gene (Derossi, et al. J. Biol. Chem. 269: 10444-10450 (1994)) and other sequences that encode peptides that mediate entry of proteins into cells as they are known or become known in the art. Furthermore, the nucleic acids of the present inventions may further include sequences that encode peptides that direct molecules to particular cellular compartments, for example, the endoplasmic reticulum or the mitochondria, as such sequences are known in the art or are later identified or developed.

The nucleic acid molecules of the present invention may be directly or indirectly labeled with a detectable marker. The detectable marker may be a radioisotope or a nonradioactive detectable molecule such as biotin or fluorescein, or other detectable markers as they are known or developed in the art. The marker may be directly or indirectly bound to the nucleic acid.

In one embodiment of the present invention, exemplified in FIG. 7, the nucleic acid molecule is a single-stranded DNA and comprises a puromycin at its 5′-end that serves as the linking moiety. Preferably, the single-stranded DNA further comprises a ribosome binding sequence and a downstream translation start codon, such as dAdTdG, and a ribosome stalling sequence such as poly(dA)ₙ or a region with strong secondary structure at the 3′-end, where poly(dA)ₙ is preferably between five and fifty dAs, more preferably between ten and forty dAs, or the secondary structure region is preferably longer than five base pairs, or more preferably, longer than ten base pairs. In this embodiment of the invention, the linking moiety is preferably at the 5′ end of the nucleic acid, and the sequences encoding the polypeptide are located between the ribosome binding sequence and ribosome stalling sequence.

In another embodiment, exemplified in FIG. 8, the nucleic acid molecule is single-stranded DNA and is labeled with a puromycin at its 3′-end that serves as the linking moiety. Preferably, the single-stranded DNA further comprises a ribosome binding sequence and a downstream translation start codon, such as dAdTdG, and a ribosome stalling sequence such as poly(dA)ₙ or a region with strong secondary structure at the 3′-end. In this embodiment of the invention, the linking moiety is preferably at the 3′ end of the nucleic acid, and the sequences encoding the polypeptide are located between the ribosome binding sequence and ribosome stalling sequence. The ribosome stalling sequence can also serve as a spacer so that the linking moiety may be able to incorporated into the nascent polypeptide. Such length can be between five to 300, preferably tebn to 100 or more preferably fifteen to fifty nucleotides in length.

Constructs in Cells

The nucleic acid molecule of the present invention and complexes that include such nucleic acid molecules can also be provided in a cell. The nucleic acid molecule can be introduced into a cell using methods known in the art, such as lipofection or electroporation. In addition, nucleic acid molecules can be introduced into cells using vectors, such as viruses or phages. The cells can be any cell, including prokaryotic or eukaryotic cells, and can be ex vivo or in vivo, including within a whole organism, including a mammal, including a human. Once introduced into a cell, the nucleic acid molecule can be transcribed and/or translated to produce a complex of the present invention.

Constructs with Other Linear Nucleic Acid

The nucleic acid molecules of the present invention, including those that optionally include a sequence of interest or a random sequence, can also be provided in other linear form of nucleic acid molecule, such as double-stranded DNA and RNA and DNA/RNA duplex. If the nucleic acids provided are in these forms, they all have to be converted to single-stranded DNA so that the nucleic acid can be translated to polypeptide.

Constructs in Vectors

The nucleic acid molecules of the present invention, including those that optionally include a sequence of interest or a random sequence, can also be provided in a vector. Vectors can be viral vectors, liposomes, microspheres, plasmids, phages or a linear dsDNA molecules. Vectors preferably include double-stranded DNA molecules of the present invention, but the invention is not limited to such vectors. For example, various viral vectors include single-stranded DNA (parvoviruses), single-stranded RNA (retroviruses) or double-stranded RNA (rotaviruses). Vectors are useful for making libraries of nucleic acid molecules of the present invention, particularly libraries that include nucleic acid molecules that have different random sequences or sequences of interest. Furthermore, such vectors are convenient for making, storing and transporting nucleic acid molecules of the present invention. Vectors can be made or modified using methods in molecular biology as they are known in the art (Sambrook et al., supra, (1989)). The vector of the present invention can be of any vector as that term is known in the art.

Vectors can be any vector known in the art, for example, retroviruses (U.S. Pat. No. 5,399,346 to Anderson et al., issued Mar. 21, 1995, Bandara et al., DNA and Cell Biol. 11:227-231 (1992)); adenoviruses (Berkner, BioTechniques 6: 616-629 (1989); adeno-associated viruses (Larrick and Burck, Gene Therapy. Application of Molecular Biology, Elsevier, N.Y. (1991); plasmid vectors (U.S. Pat. No. 5,240,846 to Collins et al., issued Aug. 31, 1993); liposomes (Holmberg et al., J. Liposome Res. 1:393-406 (1990) and Liu et al., Nature Biotechnology 15:167-173 (1997)); microspheres (Mathlowitz et al., Nature 386:410-_(1997)); see generally Larrick et al., Gene Therapy, Elsevier, N.Y. (1991) and Pinkert, Transgenic Animal Technology, Academic Press, San Diego (1994).

The present invention includes a cell that includes or has been transfected or transformed by a vector of the present invention. Such a cell can be ex vivo or in vivo in a subject, including a test animal or a human. Preferably, the nucleic acid molecule of the present invention in the vector can be expressed in the cell. In one aspect of the present invention, the nucleic acid molecule includes a sequence of interest such that when translated retains at least one activity of the polypeptide encoded by the sequence of interest. In that way, the number of translated polypeptides encoded by the sequence of interest is reduced, which results in a dosing effect of the polypeptide of interest within the cell. Alternatively, the vector includes at least one random sequence. The activity of the polypeptide encoded by the random sequence in the cell can be monitored by observing or interrogating the cell using methods known in the art, including the use of reporter genes to report changes in signal transduction within a cell.

Constructs with Binding Peptides

The nucleic acid molecule of the present invention is operably linked to its encoding polypeptide. Preferably, the operable link between the nucleic acid molecule and the polypeptide of the present invention occurs with covalent bonding. A ribosome translates the ssDNA sequence directly into a polypeptide. Preferably, the linking moiety is incorporated into the growing peptide at its C-terminal.

The interactions involved in the direct linking of the linking moiety to the polypeptide can be any of interactions that result in a irreversible binding. reversible binding is characterized by covalent bond as they are known in the art.

Constructs with Random Peptides or Sequences of Interest

A nucleic acid molecule of the present invention can also be operably and covalently linked to a polypeptide encoded by a random sequence or a sequence of interest.

Operably linked in this instance refers to the case where the nucleic acid can directly or indirectly bind with the linking moiety and the polypeptide encoded by the random sequence or sequence of interest is capable of binding with a substance of interest, such as a ligand.

Complexes comprising nucleic acid molecules of the present invention operably linked to polypeptides may also be labeled with a detectable label. The detectable label may be a radioisotope or a nonradioactive detectable molecule such as biotin, or other detectable moieties as they are known or developed in the art. The label may be directly or indirectly bound to the nucleic acid of the complex or to a polypeptide of the complex, or both. The polypeptide of the complex labeled with a detectable marker need not be the polypeptide encoded by or partially encoded by the random sequence or sequence of interest.

Construct Bound with a Substance of Interest

In another aspect of the present invention, a nucleic acid molecule of the present invention can link to a polypeptide encoded by said sequence or sequence of interest. The polypeptide can form a structure to bind with a substance of interest. This aspect of the invention allows the selection of polypeptides that bind with a substance of interest, while at the same time selecting the nucleic acid molecule that encodes the polypeptide that binds with a substance of interest. The substance of interest can be on a solid support, and can be immobilized on such a solid support using methods known in the art such as absorption, chemical conjugation or cross-linking.

Alternatively, the structure formed by a nucleic acid of the present invention and a polypeptide encoded by a random sequence or sequence of interest can be immobilized on a solid support and one or more substances of interest may be bound with or capable of reacting with the fixed complex or complexes.

Furthermore, the substance of interest can be on or within a cell, and the cell can be immobilized on a solid support using appropriate methods, such as solid supports covered with fibronectin or other adhesion molecules, entrapped thereon. The cell can be ex vivo and can be provided as a cell, culture of cells, or part of a sample of tissue, fluid or organ. Alternatively, the cell can be in vivo in a subject.

The substance of interest may be a cell-type-specific or tissue-specific molecule, such that nucleic acids or peptides of the present invention that specifically bind to the substance of interest can be identified. Such cell-type-specific and tissue-specific molecules can be used to target drug delivery to specific cells or tissues.

The cell can be any cell, including a normal cell or an abnormal cell, such as, for example, a neoplastic cell or a virus infected cell. The substance of interest can also be on or within an etiological agent, such as, for example, a virus, a bacteria, a bacterial spore, a parasite or a prion. The substance of interest may be one or a plurality of molecules on or within an etiological agent, virus, bacterium, protozoan, tumor cell or abnormal cell. The substance of interest used for selection may be whole cells, viruses, or microorganisms fixed to a solid support or in solution, or may be a portion or fractionated preparation of one or more cells, viruses, or microorganisms fixed to a solid support or in solution.

The substance of interest can also include at least one organic molecule, an inorganic molecule, a polymer, a polypeptide, a lipid, a carbohydrate, a small molecule, a nucleic acid molecule, a ribozyme, a biomacromolecule or a drug.

VI Libraries

The present invention also includes a library of nucleic acid molecules. Such libraries include nucleic acid molecules contain linking moieties so that they are able to covalently link to the respective encoding polypeptides.

The library of nucleic acid molecules can include at least two different random sequences, at least two different sequences of interest or a combination of at least one random sequence and at least one sequence of interest. For example, a library of nucleic acid molecules can include two such nucleic acid molecules. Each nucleic acid molecule can have a different random sequence or a different sequences of interest. In addition, one nucleic acid molecule can have a random sequence and the other nucleic acid molecule can have a sequence of interest.

The library can be fixed to a solid support. In particular, libraries containing random sequences, which may include sequences in which one or a few sequence positions have been randomly or semi-randomly varied, or libraries containing sequences of interest, can be fixed to a chip or array for screening with one or more substances of interest. A translated library of complexes may also be fixed to a solid support. In particular, libraries of complexes containing random sequences, which may include sequences in which one or a few sequence positions have been randomly or semi-randomly varied, or libraries of complexes containing sequences of interest, can be fixed to a chip or array for screening with one or more substances of interest.

Members of the libraries of the present invention may be labeled with a detectable label. The detectable label may be a radioisotope or a nonradioactive detectable molecule such as biotin, or other detectable moieties as they are known or developed in the art. The label may be directly or indirectly bound to the members of the library. Library members may be labeled by direct or indirect binding of a detectable marker to the nucleic acid or to a polypeptide of the library member.

Library with a Substance of Interest

A library of nucleic acid molecules can also include at least one substance of interest. The substance of interest can be bound with a nucleic acid molecule, a polypeptide, or complex of the present invention, can be unbound, or can be bound to some members of the library and not bound to other members of the library. The substance of interest can be directly bound or indirectly bound to a nucleic acid molecule of the present invention, but is preferably indirectly bound to a nucleic acid molecule of the present invention or directly bound to a complex of the present invention (particularly the polypeptide encoded by the random sequence or sequence of interest). Alternatively, the substance of interest can be a substrate, such as an enzymatic substrate, with which a nucleic acid molecule, polypeptide, or complex of the present invention interacts. Reactions of the nucleic acid molecule, polypeptide, or complex of the present invention with the substance of interest may be monitored and quantitated using appropriate assays, for example spectrophotometric assays or assays that measure the release of a radioactive moiety. The substance of interest can be directly or indirectly bound on a solid support or in solution.

In an alternative format, the structure formed by the construct of the present invention and the polypeptide encoded by a random sequence or sequence of interest can be immobilized on a solid support and one or more substances of interest may be unbound, may be bound with the fixed one or more complexes of the library, or may be acted upon in a biochemical reaction catalyzed by or modulated by one or more complexes of the library.

The substance of interest can be on or within a cell, wherein the cell can be ex vivo or in vivo, such as in a subject. The cell can be any cell, including a normal cell or an abnormal cell, such as, for example, a neoplastic cell or a virus infected cell. The substance of interest can also be one or a plurality of molecules on or within an abnormal or normal cell or on or within an etiological agent, such as, for example, a virus, a bacterium, a bacterial spore, a parasite or a prion. The substance of interest may be one or a plurality of molecules on or within an etiological agent, virus, bacterium, protozoan, tumor cell or abnormal cell. The substance of interest used for selection may be whole cells, viruses, or microorganisms fixed to a solid support or in solution, or may be a portion or fractionated preparation of one or more cells, viruses, or microorganisms fixed to a solid support or in solution.

The substance of interest may be a cell-type-specific or tissue-specific molecule, such that nucleic acids or peptides of the present invention that specifically bind to the substance of interest can be identified. Such cell-type-specific and tissue-specific molecules can be used to target drug delivery to specific cells or tissues.

The substance of interest can also include at least one organic molecule, an inorganic molecule, a polymer, a polypeptide, a lipid, a carbohydrate, a small molecule, a nucleic acid molecule, a ribozyme, a biomacromolecule or a drug.

Library of Vectors

The present invention also includes a library of vectors of the present invention. Preferably, the library of vectors includes at least two different random sequences, at least two different sequences of interest, or a combination or random sequences and sequences of interest.

VII Methods for Identifying Nucleic Acid Molecules

The present invention includes methods for identifying nucleic acid molecules, particularly from a library of random sequences or sequences of interest that encode polypeptides that bind with a substance of interest.

Single-stranded DNA

This method includes: providing at least one single-stranded nucleic acid molecule of the present invention that includes at least one random sequence or at least one sequence of interest; translating the single-stranded DNA molecule to form at least one complex, wherein the complex comprises a single-stranded DNA operably linked to the its own encoding polypeptide; contacting at least one complex with at least one substance of interest; selecting at least one complex that binds with said at least one substance of interest; and identifying said random sequence or said nucleic acid sequence of interest or nucleic acid molecule of interest. The nucleic acid molecule, including the random sequence or selected sequence can be sequenced.

For example, a nucleic acid molecule of the present invention or a library thereof in the form of a complex can be made by directly translating single-stranded DNA. Single-stranded DNA can be translated under certain conditions by ribosomes that use single-stranded DNA as a template and d(ATG) as a start codon (see, for example, Morgan et al. (1967) J Mol Biol 26: 477-497; Hulen et al. (1977) Biochimie 59:179-188; Salas and Bollum, J. (1969) Biol. Chem. 244:1152-1156; Bretscher (1968) Nature 220:1088-1091; Thorpe and Ihler (1974) Biochimica et Biophysica Acta 336:235-239; Ricker and Kaji (1991) Nucleic Acids Res. 19:6573-6578). The single-stranded DNA may be made from a variety of methods that are known to the art (for example Ellington, A. D. and Szostak, J. W (1992) Nature 355, 850; Cui, Y. et al. (1995) J. Bacerial. 177, 4872; Kujau, M. J. and Wolfi, S. (1997) Mol. Biotech. 7, 333; Williams, K. P. and Bartel, D. P. (1995) Nucleic Acid Res. 23, 4220-4221; Guo, L. H. and Wu, R. (1982) Nucleic Acid Res. 10, 2065-2084).

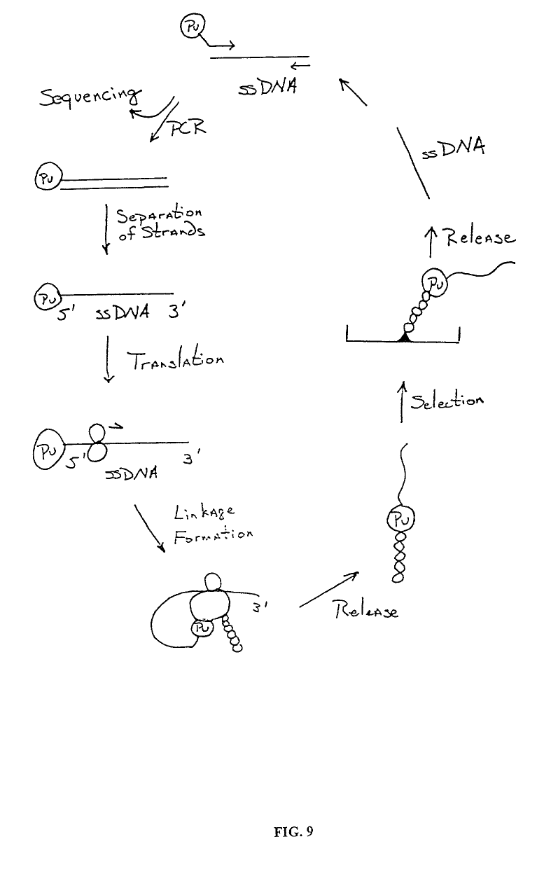

The linking moiety can be attached to either 3′- or 5′-end of the single-stranded DNA molecule using chemical and/or enzymatic synthesis. For example, if the linking moiety is at 5′-end of a single-stranded DNA as examplified in FIG. 9, a puromycin molecule can be chemically linked to an oligonucleotide at its 5′-end using appropriate chemical synthesis. The oligonucleotide can be used as one of a pair of PCR primer to synthesize double-stranded DNA. The double-stranded DNA duplex can then be dissociated or degraded to single-stranded using the methods known to the art (for example Ellington, A. D. and Szostak, J. W (1992) Nature 355, 850; Cui, Y. et al. (1995) J. Bacerial. 177, 4872; Kujau, M. J. and Wolfl, S. (1997) Mol. Biotech. 7, 333; Williams, K. P. and Bartel, D. P. (1995) Nucleic Acid Res. 23, 4220-4221; Guo, L. H. and Wu, R. (1982) Nucleic Acid Res. 10, 2065-2084). Alternatively, the chemically synthesized 5′-puromycin labeled oligodeoxyribonucleotide can be used directly the template for protein synthesis if the oligodeoxyribonucleotide contains the features as a regular messenger for protein synthesis. The single-stranded DNA is used as the template for protein translation by ribosome, and the ribosome incorporate the linking moiety, such as puromycin, into the growing peptide chain and thus forms a ssDNA-polypeptide complex. The complex is put in the mixture of target molecule or molecule of interest and the polypeptide portion of the complex binds with the target molecule or the molecule of interest. The bound complex is separated from the bound complexes. The nucleic acid on the bound complex can be eluted from the complex or be used directly as the template for nucleic amplification reactions such as PCR. The amplified nucleic acid can be sequenced or expressed in living organisms. If one of the primers is linking moiety-labeled, the amplified nucleic acid would be labeled with the linking moiety. Therefore, the linking moiety-labeled single-stranded DNA can be generated and the entire selection procedure can be repeated.

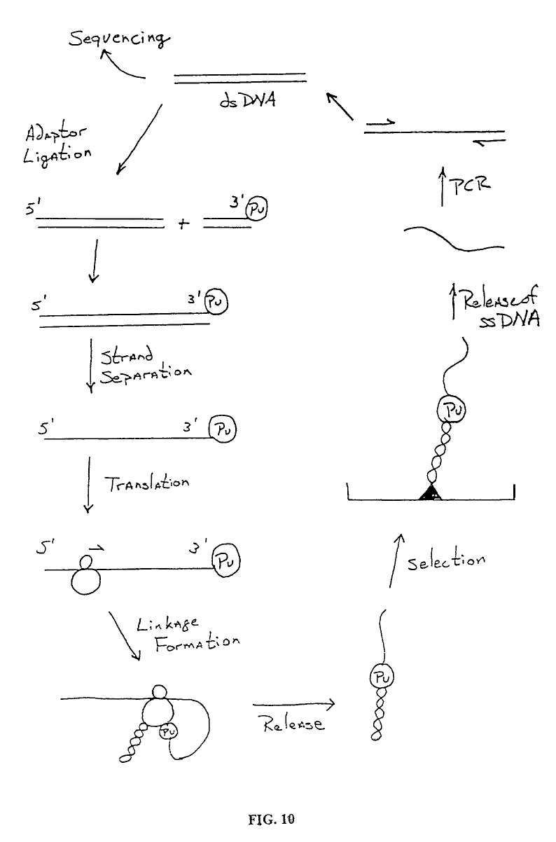

The linking moiety can also be at 3′-end of single-stranded DNA. For example (FIG. 10) a puromycin moiety can be coupled to the 3′-end of an oligonucleotide using appropriate methods such as solid phase synthesis. The single-stranded oligonucleotide can be annealed to a complimentary sequence to form a double-stranded oligonucleotide adapter. The adapter can be ligated to a double-stranded DNA with appropriate end. The adapter-ligated double-stranded DNA is converted to single-stranded DNA using methods known to the art (for example Ellington, A. D. and Szostak, J. W (1992) Nature 355, 850; Cui, Y. et al. (1995) J. Bacerial. 177, 4872; Kujau, M. J. and Wolfl, S. (1997) Mol. Biotech. 7, 333; Williams, K. P. and Bartel, D. P. (1995) Nucleic Acid Res. 23, 4220-4221; Guo, L. H. and Wu, R. (1982) Nucleic Acid Res. 10, 2065-2084). The single-stranded DNA can be used as the template for protein translation by ribosomes, and the ribosome can incorporate the linking moiety, such as puromycin, into the growing peptide chain and thus form a ssDNA-polypeptide complex. The complex can be contacted with one or more substances of interest such that the polypeptide portion of one or more complexes can bind with one or more substances of interest. Bound complexes are separated from the unbound complexes. Nucleic acid molecules of the bound complex can be eluted from the complexes or can be used directly as templates for amplification reactions such as PCR. The amplified nucleic acid molecules can be sequenced or expressed in living organisms. The PCR product can also be ligated to the double-stranded oligonucleotide adapter for a subsequent round of selection.

It may be desirable, following translation, to incubate the translation mixture under particular conditions of salt or temperature or a time period, and/or with enzymes or chemicals that may enhance the formation or stability of the complexes or may modify the complexes to enhance their efficiency in screening protocols or other applications. The complex may optionally be depleted of ribosomes by treating the mixture of translated constructs with reagents that are known to cause the dissociation of ribosomes from single-stranded DNA. For example, following translation, EDTA may be added to deplete free Mg²⁺ in the reaction mixture. This may be desirable for screening applications where the ribosome may impede binding to the substance of interest, or impede the entry of complexes into cells.

Complexes, including libraries of complexes, may be purified or substantially purified from the reaction mixture using reagents that bind parts of the complex. For example, if the polypeptide contains a stretched of adjacent six histidine residues, the single-stranded DNA-polypeptide complex may be purified from the translation reaction using nitrilotriacetic acid linked beads. Purified or substantially purified complexes may be stored under conditions that promote the stability of nucleic acids and polypeptides, for example, at 4° C. in a buffer that contains BSA and EDTA.

Optionally, the single-stranded nucleic acid in said complex can be converted to double-stranded DNA using the methods known in the art, such as using T4 DNA polymerase.

The complex is contacted with one or more substances of interest under conditions that promote the binding of the complex, or reaction of the complex, particularly the polypeptide encoded by the random sequence or sequence of interest, with the substance of interest. The substance of interest can be on a solid support or in solution. A solid support may be a chip or array. The substance of interest can be on or within a cell and can be on or within an etiological agent. Thus, a substance of interest is bound with a complex that includes a polypeptide encoded by a random sequence or a sequence of interest and the random sequence or sequence of interest itself. Complexes that are not bound to a substance of interest can be separated from bound complexes using methods known in the art. For example, if the substance of interest is bound on a solid support and complexes are bound to the substance of interest or free in solution, the complexes that are free in solution can be washed away using methods known in the art for receptor-ligand reactions, such as immunoassay methods. Alternatively, the complexes of the present invention may be fixed to a chip or array, and the substance or substances of interest may be contacted with the chip or array to allow the substance of interest to bind or react with complexes for which the substance of interest has affinity. The substance of interest may be labeled with a detectable marker, or may be detected with a reagent specific for the substance of interest. Nonspecifically bound substance of interest may be washed off using appropriate methods as they are known in the art prior to detection of the bound substance of interest. Thus, the nucleic acid molecule encoding a peptide that binds with or reacts with a substance of interest has been selected using this method.

The selected complex or portions thereof, such as the polypeptide or the nucleic acid molecule encoding the polypeptide, can be isolated by recovery using a variety of methods. For example, changes in pH, detergents, denaturing agents (such as phenol, urea or guanidinium), concentration and types of salts, such as chaotropic or anti-chaotropic salts, or combinations thereof can be used to elute the complex or portions thereof. Alternatively, the complex can be digested using enzymes, such as proteases or nucleases to free portions of the complex such as the polypeptide or the nucleic acid molecule.

The bound nucleic acid is recovered and enriched using appropriate nucleic amplification reactions, such as polymerase chain reaction. The enriched nucleic acid can be sequenced using appropriate methods known to the art.

In other applications where the selection requires multiple rounds of selection, the enriched nucleic acid can be converted to single-stranded DNA again and used as the template for protein synthesis. If the linking moiety is at 5′ of the translation template, the single-strand converted to single-stranded DNA directly from the double-stranded DNA. If the linking moiety needs to be at 3′ of the translating template, a linking moiety-attached adapter nucleic acid is needed so that the linking moiety can be ligated to the double-stranded DNA via the adapter. Then the adapter-linked double-stranded DNA can be converted to single stranded DNA using the methods known to the art (for example Ellington, A. D. and Szostak, J. W (1992) Nature 355, 850; Cui, Y. et al. (1995) J. Bacerial. 177, 4872; Kujau, M. J. and Wolfi, S. (1997) Mol. Biotech. 7, 333; Williams, K. P. and Bartel, D. P. (1995) Nucleic Acid Res. 23, 4220-4221; Guo, L. H. and Wu, R. (1982) Nucleic Acid Res. 10, 2065-2084).

The recovered nucleic acid molecules that contain a random sequence or sequence of interest can be cloned into appropriate vectors, such as plasmids, which can be amplified in an appropriate host. The recovered nucleic acid, which may contain several DNA species, may also be separated using the methods that exploit the sequence and conformation of the nucleic acid. It may be necessary to separate the double-stranded PCR product to single-stranded in order to use the said method. These methods can be capillary affinity gel for nucleic acid and HPLC, or the combination thereof. The individual species of the nucleic acid molecules can then be sequenced. The amino acid sequence of the polypeptide that is able to bind to the substance of interest can be deduced from the nucleic acid sequence.

The entire selection procedures, or portions thereof, may be automated. Translated complexes can be contacted with targets and unbound complexes may be washed away by a programmable machine. Another component of the automated machine may perform amplification reactions on the nucleic acid molecules of the bound complexes. Several rounds of selection and amplification may be automated in a linked process, and the final PCR products can be separated on a column that utilizing the difference in sequence and conformation. Individual nucleic acid molecules may be transmitted directly to an automated sequencer and sequenced, for example using fluorescently tagged nucleotides that may be read spectrophotometrically.

The present invention includes nucleic acid molecules that comprise at least a portion of a random sequence or selected nucleic acid sequence identified by this method. The present invention also includes polypeptides that include at least a portion of a polypeptide encoded by an identified random sequence or sequence of interest.

Other Forms of Nucleic Acid Molecules

The present invention also includes a method for identifying a nucleic acid molecule or sequence in other forms, which include double-stranded DNA, single- or double-stranded RNA or RNA-DNA duplex. The sources for these forms of nucleic acid can be either synthetic or biological such as viral, prokaryotic and eukaryotic. All these nucleic acid must be directly or indirectly converted to single-stranded DNA as the template for protein synthesis in order to form a polypeptide-single-stranded DNA complex in this invention.

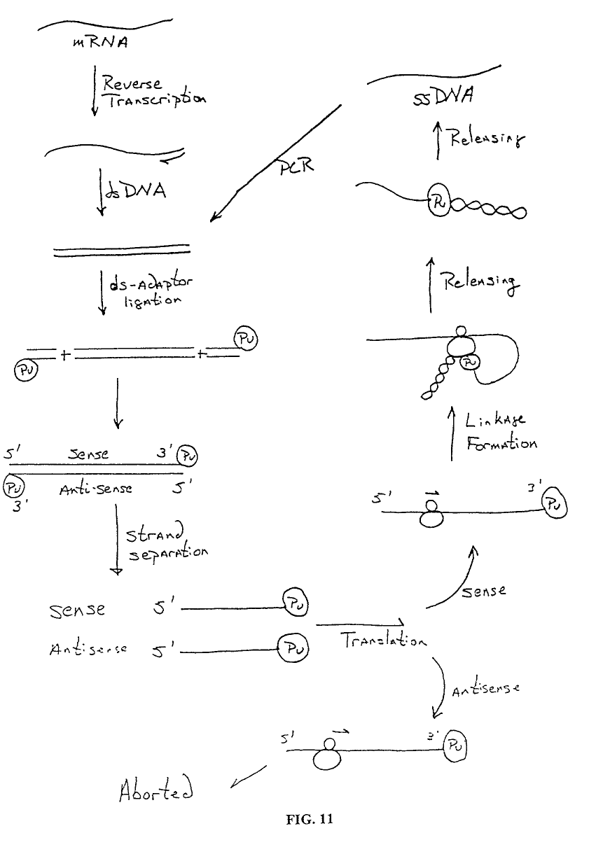

For example (FIG. 11), total eukaryotic cellular mRNA may be converted to double-stranded DNA using the methods known to the art. The single-stranded DNA corresponding to the mRNA sequences can be ligated with a linking moiety. Then, the selection and enrichment of the nucleic acid sequences can be carried out using the procedures described in the previous section.

The present invention includes nucleic acid molecules that include at least a portion of a random sequence or selected nucleic acid sequence identified by this method. The present invention also includes polypeptides that include at least a portion of a polypeptide encoded by an identified random sequence or sequence of interest.

VIII Methods for Identifying Polypeptides

The present invention includes methods for identifying nucleic polypeptides, particularly polypeptides encoded by random sequences or sequences of interest that bind with a substance of interest.

Single-stranded DNA

This method includes: providing at least one nucleic acid molecule of the present invention as a single-stranded DNA that includes at least one random sequence or at least one sequence of interest; translating the single-stranded DNA molecule to form at least one complex, wherein the complex comprises a single-stranded DNA operably linked to the its own encoding polypeptide; contacting at least one complex with at least one substance of interest; selecting at least one complex that binds with said at least one substance of interest; and identifying said random sequence or said nucleic acid sequence of interest or nucleic acid molecule of interest. The nucleic acid molecule, including the random sequence or selected sequence can be sequenced.

For example, a nucleic acid molecule of the present invention or a library thereof in the form of a complex can be made by directly translating single-stranded DNA. Single-stranded DNA can be translated under certain conditions by ribosomes that use single-stranded DNA as a template and d(ATG) as a start codon (see, for example, Morgan et al. (1967) J Mol Biol 26: 477-497; Hulen et al. (1977) Biochimie 59:179-188; Salas and Bollum, J. (1969) Biol. Chem. 244:1152-1156; Bretscher (1968) Nature 220:1088-1091; Thorpe and Ihler (1974) Biochimica et Biophysica Acta 336:235-239; Ricker and Kaji (1991) Nucleic Acids Res. 19:6573-6578). The single-stranded DNA may be made from a variety of methods that are known to the art (for example Ellington, A. D. and Szostak, J. W (1992) Nature 355, 850; Cui, Y. et al. (1995) J. Bacerial. 177, 4872; Kujau, M. J. and Wolfl, S. (1997) Mol. Biotech. 7, 333; Williams, K. P. and Bartel, D. P. (1995) Nucleic Acid Res. 23, 4220-4221; Guo, L. H. and Wu, R. (1982) Nucleic Acid Res. 10, 2065-2084).

The linking moiety can be attached to either 3′- or 5′-end of the single-stranded DNA molecule using chemical and/or enzymatic synthesis. For example, if the linking moiety is at 5′-end of a single-stranded DNA as exemplified in FIG. 9, a puromycin molecule can be chemically linked to an oligonucleotide at its 5′-end using appropriate chemical synthesis. The oligonucleotide can be used as one of a pair of PCR primer to synthesize double-stranded DNA. The double-stranded DNA duplex can then be dissociated or degraded to single-stranded using the methods known to the art (for example Ellington, A. D. and Szostak, J. W (1992) Nature 355, 850; Cui, Y. et al. (1995) J. Bacerial. 177, 4872; Kujau, M. J. and Wolfl, S. (1997) Mol. Biotech. 7, 333; Williams, K. P. and Bartel, D. P. (1995) Nucleic Acid Res. 23, 4220-4221; Guo, L. H. and Wu, R. (1982) Nucleic Acid Res. 10, 2065-2084). Alternatively, the chemically synthesized 5′-puromycin labeled oligodeoxyribonucleotide can be used directly the template for protein synthesis. The linking moiety can also be linked to 3′-end of single-stranded DNA. For example (FIG. 10), a puromycin can be labeled to the 3′-end of an oligonucleotide using appropriate chemical synthesis method. The single-stranded oligonucleotide can be annealed to a complimentary sequence to form a double-stranded oligonucleotide adapter. The adapter can be ligated to a double-stranded DNA with appropriate end. The adapter-ligated double-stranded DNA is converted to single-stranded DNA using the method known to the art (for example Ellington, A. D. and Szostak, J. W (1992) Nature 355, 850; Cui, Y. et al. (1995) J. Bacerial. 177, 4872; Kujau, M. J. and Wolfi, S. (1997) Mol. Biotech. 7, 333; Williams, K. P. and Bartel, D. P. (1995) Nucleic Acid Res. 23, 4220-4221; Guo, L. H. and Wu, R. (1982) Nucleic Acid Res. 10, 2065-2084).

Following translation, it may be desirable to incubate the translation mixture under particular conditions of salt or temperature or a time period, and/or with enzymes or chemicals that may enhance the formation or stability of the complexes or may modify the complexes to enhance their efficiency in screening protocols or other applications. The complex may optionally be depleted of ribosomes by treating the mixture of translated constructs with reagents that are known to cause the dissociation of ribosomes from single-stranded DNA. For example, following translation, EDTA may be added to deplete free Mg²⁺ in the reaction mixture. This may be desirable for screening applications where the ribosome may impede binding to the substance of interest, or impede the entry of complexes into cells.

Complexes, including libraries of complexes, may be purified or substantially purified from the reaction mixture using reagents that bind parts of the complex. For example, if the polypeptide contains a stretched of adjacent six histidine residues, the single-stranded DNA-polypeptide complex may be purified from the translation reaction using nitrilotriacetic-linked beads. Purified or substantially purified complexes may be stored under conditions that promote the stability of nucleic acids and polypeptides, for example, at 4° C. in a buffer that contains BSA and EDTA.

Optionally, the single-stranded nucleic acid in said complex can be converted to double-stranded DNA using the methods known in the art, such as using T4 DNA polymerase.

The complex is contacted with one or more substances of interest under conditions that promote the binding of the complex, or reaction of the complex, particularly the polypeptide encoded by the random sequence or sequence of interest, with the substance of interest. The substance of interest can be on a solid support or in solution. A solid support may be a chip or array. The substance of interest can be on or within a cell and can be on or within an etiological agent. Thus, a substance of interest is bound with a complex that includes a polypeptide encoded by a random sequence or a sequence of interest and the random sequence or sequence of interest itself. Complexes that are not bound to a substance of interest can be separated from bound complexes using methods known in the art. For example, if the substance of interest is bound on a solid support and complexes are bound to the substance of interest or free in solution, the complexes that are free in solution can be washed away using methods known in the art for receptor-ligand reactions, such as immunoassay methods. Alternatively, the complexes of the present invention may be fixed to a chip or array, and the substance or substances of interest may be contacted with the chip or array to allow the substance of interest to bind or react with complexes for which the substance of interest has affinity. The substance of interest may be labeled with a detectable marker, or may be detected with a reagent specific for the substance of interest. Nonspecifically bound substance of interest may be washed off using appropriate methods as they are known in the art prior to detection of the bound substance of interest. Thus, the nucleic acid molecule encoding a peptide that binds with or reacts with a substance of interest has been selected using this method.

The selected complex or portions thereof, such as the polypeptide or the nucleic acid molecule encoding the polypeptide, can be isolated by recovery using a variety of methods. For example, changes in pH, detergents, denaturing agents (such as phenol, urea or guanidinium), concentration and types of salts, such as chaotropic or anti-chaotropic salts, or combinations thereof can be used to elute the complex or portions thereof. Alternatively, the complex can be digested using enzymes, such as proteases or nucleases to free portions of the complex such as the polypeptide or the nucleic acid molecule.

The bound nucleic acid is recovered and enriched using appropriate nucleic amplification reactions, such as polymerase chain reaction. The enriched nucleic acid can be sequenced using appropriate methods known to the art.

In other applications where the selection requires multiple rounds of selection, the enriched nucleic acid can be converted to single-stranded DNA again and used as the template for protein synthesis. If the linking moiety is at 5′ of the translation template, the single-strand converted to single-stranded DNA directly from the double-stranded DNA. If the linking moiety needs to be at 3′ of the translating template, a linking moiety-attached adapter nucleic acid is needed so that the linking moiety can be ligated to the double-stranded DNA via the adapter. Then the adapter-linked double-stranded DNA can be converted to single stranded DNA using the methods known to the art (for example Ellington, A. D. and Szostak, J. W (1992) Nature 355, 850; Cui, Y. et al. (1995) J. Bacerial. 177, 4872; Kujau, M. J. and Wolfi, S. (1997) Mol. Biotech. 7, 333; Williams, K. P. and Bartel, D. P. (1995) Nucleic Acid Res. 23, 4220-4221; Guo, L. H. and Wu, R. (1982) Nucleic Acid Res. 10, 2065-2084).

The recovered nucleic acid molecules that contain a random sequence or sequence of interest can be cloned into appropriate vectors, such as plasmids, which can be amplified in an appropriate host. The recovered nucleic acid, which may contain several DNA species, may also be separated using the methods that exploit the sequence and conformation of the nucleic acid. It may be necessary to separate the double-stranded PCR product to single-stranded in order to use the said method. These methods can be capillary affinity gel for nucleic acid and HPLC, or the combination thereof. The individual species of the nucleic acid molecules can then be sequenced. The amino acid sequence of the polypeptide that is able to bind to the substance of interest can be deduced from the nucleic acid sequence.

The entire selection procedures, or portions thereof, may be automated. Translated complexes can be contacted with targets and unbound complexes may be washed away by a programmable machine. Another component of the automated machine may perform amplification reactions on the nucleic acid molecules of the bound complexes. Several rounds of selection and amplification may be automated in a linked process, and the final PCR products can be separated on a column that utilizing the difference in sequence and conformation. Individual nucleic acid molecules may be transmitted directly to an automated sequencer and sequenced, for example using fluorescently tagged nucleotides that may be read spectrophotometrically.

The present invention includes nucleic acid molecules that comprise at least a portion of a random sequence or selected nucleic acid sequence identified by this method. The present invention also includes polypeptides that include at least a portion of a polypeptide encoded by an identified random sequence or sequence of interest.

The sequence of the polypeptides that bind to the target molecule or molecule of interest can be deduced from the nucleic acid sequence. The polypeptide may be obtained by expressing the genes in appropriate organisms or chemical synthesis. The obtained polypeptide may be assayed for its binding or biological activity.

Other Forms of Nucleic Acid Molecules

The present invention also includes a method for identifying a nucleic acid molecule or sequence in other forms, which include double-stranded DNA, single- or double-stranded RNA or RNA-DNA duplex. However, all these nucleic acid must be directly or indirectly converted to single-stranded DNA in order to form a polypeptide-single-stranded DNA complex in this invention.

The sequence of the polypeptides that bind to the target molecule or molecule of interest may be deduced from the nucleic acid sequence. The polypeptide may be obtained by expressing the genes in appropriate organisms or chemical synthesis. The obtained polypeptide may be assayed for its binding or biological activity.

IX Methods for Identifying Test Compounds

The present invention includes methods for identifying test compounds, test compounds identified by this method and pharmaceutical compositions identified by this method.

One aspect of the present invention is a method for identifying a test compound, including: 1) contacting a target with a complex that comprises a nucleic acid molecule that comprises an open reading frame, a linking moiety, and a polypeptide encoded, at least in part, by the open reading frame, wherein the open reading frame comprises a random sequence or sequence of interest, and wherein the linking moiety is directly or indirectly bound to the nucleic acid molecule and to the polypeptide; 2) identifying complexes bound with said target, or identifying complexes on the basis of catalytic function or the results of cellular assays; determining the structure of the polypeptide encoded by the random sequence or sequence of interest; and 3) identifying moieties that have structures that have space filling shapes that are similar to at least a portion of said identified moiety. The present invention also includes a test compound identified by this method and a pharmaceutical composition identified by this method.

Complexes, nucleic acid molecules and polypeptides of the present invention that bind with a substance of interest, such as a target, including a pharmaceutical target, or complexes that comprise peptides or nucleic acids with desirable catalytic properties, can be identified using methods of the present invention. The structure of the identified nucleic acid molecule or amino acid can be determined using methods such as NMR and mass spectroscopy. Alternatively, the identified nucleic acid molecule sequences or amino acid sequences can be provided to a processing unit and appropriate computer models and software to model the three dimensional configuration of the peptide that binds the target encoded therein. Appropriate computer models and software can also provide structures of chemical libraries that correspond to at least a portion of the three dimensional configuration. These chemical libraries can be synthesized in whole or in part by combinatorial chemistry methodologies. These libraries can then be screened for activity, such as pharmaceutical activity, using methods known in the art and described herein.

Pharmacology and Toxicity of Test Compounds

The structure of a test compound can be determined or confirmed by methods known in the art, such as mass spectroscopy. For test compounds stored for extended periods of time under a variety of conditions, the structure, activity and potency thereof can be confirmed.

Identified test compounds can be evaluated for a particular activity using are-recognized methods and those disclosed herein. For example, if an identified test compound is found to have anticancer cell activity in vitro, then the test compound would have presumptive pharmacological properties as a chemotherapeutic to treat cancer. Such nexuses are known in the art for several disease states, and more are expected to be discovered over time. Based on such nexuses, appropriate confirmatory in vitro and in vivo tests of pharmacological activity, and toxicology, and be selected and performed. The methods described herein can also be used to assess pharmacological selectivity and specificity, and toxicity.

Identified test compounds can be evaluated for toxicological effects using known methods (see, Lu, Basic Toxicology, Fundamentals, Target Organs, and Risk Assessment, Hemisphere Publishing Corp., Washington (1985); U.S. Pat. No.; 5,196,313 to Culbreth (issued Mar. 23, 1993) and U.S. Pat. No. 5,567,952 to Benet (issued Oct. 22, 1996)). For example, toxicology of a test compound can be established by determining in vitro toxicity towards a cell line, such as a mammalian, for example human, cell line. Test compounds can be treated with, for example, tissue extracts, such as preparations of liver, such as microsomal preparations, to determine increased or decreased toxicological properties of the test compound after being metabolized by a whole organism. The results of these types of studies are predictive of toxicological properties of a chemical in animals, such as mammals, including humans.

Alternatively, or in addition to these in vitro studies, the toxicological properties of a test compound in an animal model, such as mice, rats, rabbits, dogs or monkeys, can be determined using established methods (see, Lu, supra (1985); and Creasey, Drug Disposition in Humans, The Basis of Clinical Pharmacology, Oxford University Press, Oxford (1979)). Depending on the toxicity, target organ, tissue, locus and presumptive mechanism of the test compound, the skilled artisan would not be burdened to determine appropriate doses, LD₅₀ values, routes of administration and regimes that would be appropriate to determine the toxicological properties of the test compound. In addition to animal models, human clinical trials can be performed following established procedures, such as those set forth by the United States Food and Drug Administration (USFDA) or equivalents of other governments. These toxicity studies provide the basis for determining the efficacy of a test compound in vivo.

Efficacy of Test Compounds

Efficacy of a test compound can be established using several art recognized methods, such as in vitro methods, animal models or human clinical trials (see, Creasey, supra (1979)). Recognized in vitro models exist for several diseases or conditions. For example, the ability of a test compound to extend the life-span of HIV-infected cells in vitro is recognized as an acceptable model to identify chemicals expected to be efficacious to treat HIV infection or AIDS (see, Daluge et al., Antimicro. Agents Chemother. 41:1082-1093 (1995)). Furthermore, the ability of cyclosporin A (CsA) to prevent proliferation of T-cells in vitro has been established as an acceptable model to identify chemicals expected to be efficacious as immunosuppressants (see, Suthanthiran et al., supra (1996)). For nearly every class of therapeutic, disease or condition, an acceptable in vitro or animal model is available. The skilled artisan is armed with a wide variety of such models as they are available in the literature or from the USFDA or the National Institutes of Health (NIH). In addition, these in vitro methods can use tissue extracts, such as preparations of liver, such as microsomal preparations, to provide a reliable indication of the effects of metabolism on a test compound. Similarly, acceptable animal models can be used to establish efficacy of test compounds to treat various diseases or conditions. For example, the rabbit knee is an accepted model for testing agents for efficacy in treating arthritis (see, Shaw and Lacy, J. Bone Joint Surg. (Br.) 55:197-205 (1973)). Hydrocortisone, which is approved for use in humans to treat arthritis, is efficacious in this model which confirms the validity of this model (see, McDonough, Phys. Ther. 62:835-839 (1982)). When choosing an appropriate model to determine efficacy of test compounds, the skilled artisan can be guided by the state of the art, the USFDA or the NIH to choose an appropriate model, doses and route of administration, regime and endpoint and as such would not be unduly burdened.

In addition to animal models, human clinical trials can be used to determine the efficacy of test compounds. The USFDA, or equivalent governmental agencies, have established procedures for such studies.

Selectivity of Test Compounds

The in vitro and in vivo methods described above also establish the selectivity of a candidate modulator. It is recognized that chemicals can modulate a wide variety of biological processes or be selective. Panels of cells as they are known in the art can be used to determine the specificity of the a test compound (WO 98/13353 to Whitney et al., published Apr. 2, 1998). Selectivity is evident, for example, in the field of chemotherapy, where the selectivity of a chemical to be toxic towards cancerous cells, but not towards non-cancerous cells, is obviously desirable. Selective modulators are preferable because they have fewer side effects in the clinical setting. The selectivity of a test compound can be established in vitro by testing the toxicity and effect of a test compound on a plurality of cell lines that exhibit a variety of cellular pathways and sensitivities. The data obtained form these in vitro toxicity studies can be extended to animal model studies, including human clinical trials, to determine toxicity, efficacy and selectivity of a test compound.

The selectivity, specificity and toxicology, as well as the general pharmacology, of a test compound can be often improved by generating additional test compounds based on the structure/property relationship of a test compound originally identified as having activity. Test compounds can be modified to improve various properties, such as affinity, life-time in blood, toxicology, specificity and membrane permeability. Such refined test compounds can be subjected to additional assays as they are known in the art or described herein. Methods for generating and analyzing such compounds or compositions are known in the art, such as U.S. Pat. No. 5,574,656 to Agrafiotis et al.

Pharmaceutical Compositions

The present invention also encompasses a test compound in a pharmaceutical composition comprising a pharmaceutically acceptable carrier prepared for storage and preferably subsequent administration, which have a pharmaceutically effective amount of the test compound in a pharmaceutically acceptable carrier or diluent. Acceptable carriers or diluents for therapeutic use are well known in the pharmaceutical art, and are described, for example, in Remington's Pharmaceutical Sciences, Mack Publishing Co., (A. R. Gennaro edit. (1985)). Preservatives, stabilizers, dyes and even flavoring agents can be provided in the pharmaceutical composition. For example, sodium benzoate, sorbic acid and esters of p-hydroxybenzoic acid can be added as preservatives. In addition, antioxidants and suspending agents can be used.

The test compounds of the present invention can be formulated and used as tablets, capsules or elixirs for oral administration; suppositories for rectal administration; sterile solutions, suspensions or injectable administration; and the like. Injectables can be prepared in conventional forms either as liquid solutions or suspensions, solid forms suitable for solution or suspension in liquid prior to injection, or as emulsions. Suitable excipients are, for example, water, saline, dextrose, mannitol, lactose, lecithin, albumin, sodium glutamate, cysteine hydrochloride and the like. In addition, if desired, the injectable pharmaceutical compositions can contain minor amounts of nontoxic auxiliary substances, such as wetting agents, pH buffering agents and the like. If desired, absorption enhancing preparation, such as liposomes, can be used.

The pharmaceutically effective amount of a test compound required as a dose will depend on the route of administration, the type of animal or patient being treated, and the physical characteristics of the specific animal under consideration. The dose can be tailored to achieve a desired effect, but will depend on such factors as weight, diet, concurrent medication and other factors which those skilled in the medical arts will recognize. In practicing the methods of the present invention, the pharmaceutical compositions can be used alone or in combination with one another, or in combination with other therapeutic or diagnostic agents. These products can be utilized in vivo, preferably in a mammalian patient, preferably in a human, or in vitro. In employing them in vivo, the pharmaceutical compositions can be administered to the patient in a variety of ways, including parenterally, intravenously, subcutaneously, intramuscularly, colonically, rectally, nasally or intraperiotoneally, employing a variety of dosage forms. Such methods can also be used in testing the activity of test compounds in vivo.

As will be readily apparent to one skilled in the art, the useful in vivo dosage to be administered and the particular mode of administration will vary depending upon the age, weight and type of patient being treated, the particular pharmaceutical composition employed, and the specific use for which the pharmaceutical composition is employed. The determination of effective dosage levels, that is the dose levels necessary to achieve the desired result, can be accomplished by one skilled in the art using routine methods as discussed above, and can be guided by agencies such as the USFDA or NIH. Typically, human clinical applications of products are commenced at lower dosage levels, with dosage level being increased until the desired effect is achieved. Alternatively, acceptable in vitro studies can be used to establish useful doses and routes of administration of the test compounds.

In non-human animal studies, applications of the pharmaceutical compositions are commenced at higher dose levels, with the dosage being decreased until the desired effect is no longer achieved or adverse side effects are reduced of disappear. The dosage for the test compounds of the present invention can range broadly depending upon the desired affects, the therapeutic indication, route of administration and purity and activity of the test compound. Typically, dosages can be between about 1 ng/kg and about 10 mg/kg, preferably between about 10 ng/kg and about 1 mg/kg, more preferably between about 100 ng/kg and about 100 micrograms/kg, and most preferably between about 1 microgram/kg and about 10 micrograms/kg.

The exact formulation, route of administration and dosage can be chosen by the individual physician in view of the patient's condition (see, Fingle et al., in The Pharmacological Basis of Therapeutics (1975)). It should be noted that the attending physician would know how to and when to terminate, interrupt or adjust administration due to toxicity, organ dysfunction or other adverse effects. Conversely, the attending physician would also know to adjust treatment to higher levels if the clinical response were not adequate. The magnitude of an administrated does in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight and response of the individual patient, including those for veterinary applications.

Depending on the specific conditions being treated, such pharmaceutical compositions can be formulated and administered systemically or locally. Techniques for formation and administration can be found in Remington's Pharmaceutical Sciences, 18th Ed., Mack Publishing Co., Easton, Pa. (1990). Suitable routes of administration can include oral, nasal, rectal, transdermal, otic, ocular, vaginal, transmucosal or intestinal administration; parenteral delivery, including intramuscular, subcutaneous, intramedullary injections, as well as intrathecal, direct intraventricular, intravenous, intraperitoneal, intranasal, or intraocular injections.

For injection, the pharmaceutical compositions of the present invention can be formulated in aqueous solutions, preferably in physiologically compatible buffers such as Hanks' solution, Ringer's solution or physiological saline buffer. For such transmucosal administration, penetrans appropriate to the barrier to be permeated are used in the formulation. Such penetrans are generally known in the art. Use of pharmaceutically acceptable carriers to formulate the pharmaceutical compositions herein disclosed for the practice of the invention into dosages suitable for systemic administration is within the scope of the invention. With proper choice of carrier and suitable manufacturing practice, the compositions of the present invention, in particular, those formulation as solutions, can be administered parenterally, such as by intravenous injection. The pharmaceutical compositions can be formulated readily using pharmaceutically acceptable carriers well known in the art into dosages suitable for oral administrations. Such carriers enable the test compounds of the invention to be formulated as tables, pills, capsules, liquids, gels, syrups, slurries, suspensions and the like, for oral ingestion by a patient to be treated.

Agents intended to be administered intracellularly may be administered using techniques well known to those of ordinary skill in the art. For example, such agents may be encapsulated into liposomes, then administered as described above. Intracellular delivery of drugs may be acheived by linking peptides such as the translocating domain of the tat protein of HIV to the agent. Linkage of hydrophobic molecules such as biotin to the attached tat peptide or similar translocating peptides may improve intracellular delivery further (Chen et al. Analyt. Biochem. 227: 168-175 (1995)).Substantially all molecules present in an aqueous solution at the time of liposome formation are incorporated into or within the liposomes thus formed. The liposomal contents are both protected from the external micro-environment and, because liposomes fuse will cell membranes, are efficiently delivered into the cell cytoplasm. Additionally, due to their hydrophobicity, small organic molecules can be directly administered intracellularly.

Pharmaceutical compositions suitable for use in the present invention include compositions wherein the active ingredients are contained in an effective amount to achieve its intended purpose. Determination of the effective amount of a pharmaceutical composition is well within the capability of those skilled in the art, especially in light of the detailed disclosure provided herein. In addition to the active ingredients, these pharmaceutical compositions can contain suitable pharmaceutically acceptable carriers comprising excipients and auxiliaries which facilitate processing of the active chemicals into preparations which can be used pharmaceutically. The preparations formulated for oral administration may be in the form of tables, dragees, capsules or solutions. The pharmaceutical compositions of the present invention can be manufactured in a manner that is itself known, for example by means of conventional mixing, dissolving, granulating, dragee-making, emulsifying, encapsulating, entrapping or lyophilizing processes. Pharmaceutical formulations for parenteral administration include aqueous solutions of active chemicals in water-soluble form.

Additionally, suspensions of the active chemicals may be prepared as appropriate oily injection suspensions. Suitable lipophilic solvents or vehicles include fatty oils such as sesame oil, or synthetic fatty acid esters, such as ethyl oleate or triglycerides or liposomes. Aqueous injection suspensions may contain substances what increase the viscosity of the suspension, such as sodium carboxymethyl cellulose, sorbitol or dextran. Optionally, the suspension can also contain suitable stabilizers or agents that increase the solubility of the chemicals to allow for the preparation of highly concentrated solutions.

Pharmaceutical compositions for oral use can be obtained by combining the active chemicals with solid excipient, optionally grinding a resulting mixture, and processing the mixture of granules, after adding suitable auxiliaries, if desired, to obtain tables or dragee cores. Suitable excipients are, in particular, fillers such as sugars, including lactose, sucrose, mannitol or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose and/or polyvinylpyrrolidone. If desired, disintegrating agents can be added, such as the cross-linked polyvinyl pyrolidone, agar, alginic acid or a salt thereof such as sodium alginate. Dragee cores can be provided with suitable coatings. Dyes or pigments can be added to the tablets or dragee coatings for identification or to characterize different combinations of active doses.

The test compounds of the present invention, and pharmaceutical compositions that include such test compounds are useful for treating a variety of ailments in a patient, including a human. As set forth in the Examples, the test compounds of the present invention have antibacterial, antimicrobial, antiviral, anticancer cell, antitumor and cytotoxic activity. A patient in need of such treatment can be provided a test compound of the present invention, preferably in a pharmacological composition in an effective amount to reduce the number or growth rate of bacteria, microbes, cancer cells or tumor cells in said patent, or to reduce the infectivity of viruses in said patient. The amount, dosage, route of administration, regime and endpoint can all be determined using the procedures described herein or by appropriate government agencies, such as the United Stated Food and Drug Administration.

X Methods for Identifying Targets

The present invention includes methods for identifying targets such as pharmaceutical targets, purification targets or diagnostic targets. The present invention also includes targets and pharmaceutical targets identified by such methods.

Another aspect of the present invention are methods for identifying a target, such as a pharmaceutical target, that include: contacting a substance of interest with a complex that: comprises an open reading frame, a linking moiety, and a polypeptide encoded, at least in part, by the open reading frame, wherein the open reading frame comprises a random sequence or sequence of interest, and wherein the linking moiety is directly or indirectly bound to the nucleic acid molecule and to the polypeptide; 2) identifying complexes bound with said target, or identifying complexes on the basis of catalytic function or the results of cellular assays; determining the sequence of the polypeptide encoded by the random sequence or sequence of interest. The present invention also includes a target identified by this method, including pharmaceutical targets.

In this application of the invention, complexes comprising polypeptides that bind the etiological agent are selected, and the nucleic acids of the complexes are recovered and amplified. The individual species of the PCR product can be sequenced and the polypeptide sequence is deduced from the nucleic acid sequence. All the polypeptide can be synthesized using the deduced sequences, preferably by solid phase synthesis. The each peptide or as a combination is assayed to determine whether the peptide or peptides have desired biological effect, such as inhibit infectivity, on the etiological agent. The selected peptides that show desired biological effect on the etiological agent can be used as the probe to screen the phage cDNA library derived from the RNA of the etiological agent. The phages that are selected by the probes may contain the genes or part of the genes that is responsible for the infectivity of the etiological agent, which can be used as the potential drug target.

In addition, the ability of a complex to modulate signal transduction pathways can be determined. The ability of a complex to modulate an identified signal transduction pathways identifies such signal transduction pathway as a therapeutic target. A variety of cells that comprise reporter genes that report an increased or decreased activity of a signal transduction pathway in response to a compound are known in the art. Such cells can also be made using methods known in the art (see, WO 98/13353 to Whitney, published Apr. 2, 1999; U.S. Pat. No. 5,298,429 to Evans et al., issued Mar. 29, 1994; and Skames et al., Genes and Development 6:903-918 (1992)). Complexes of the present invention can be contacted with such cells and the expression of the reporter gene monitored to identify signal transduction pathways modulated by the complex. Such identified signal transduction pathways are themselves pharmaceutical targets, as are the individual components of the identified signal transduction pathway.

Peptides encoded by random sequences or sequences of interest may also be selected for desireable catalytic functions. Assays may be developed in which enhanced or altered function of peptides of the present invention is detectable, for example colorometric assays or assays that measure the release of radioactive moieties from substrates.

Intracellular and in vitro assays may be done in appropriate formats, such as in microtiter dishes and using plate readers. The complexes selected by such assays or portions thereof can be isolated using various purification methods and amplification methods as they are known in the art. For example, if the cellular or in vitro assay is performed in a microtiter format, complexes may be recovered from positively screening assay wells using antibodies or nucleic acids of complexes may be recovered from positively screening assay wells by amplification reactions using specific primers. Detergents, denaturing agents, and partial purification steps such as centrifugation may be used prior to recovery of the complexes or their components.

All publications, including patent documents, scientific articles and www sites, referred to in this application are incorporated by reference in their entirety for all purposes to the same extent as if each individual publication were individually incorporated by reference.

All headings are for the convenience of the reader and should not be used to limit the meaning of the text that follows the heading, unless so specified.

### BIBLIOGRAPHY

U.S. Pat. No. 5,260,163 to Gold et al., issued Dec. 14, 1993.

U.S. Pat. No. 5,279,936 to Vorpahl, issued Jan. 18, 1994.

U.S. Pat. No. 5,324,637

U.S. Pat. No. 5,643,768 to Kawasaki, issued Jul. 1, 1997.

U.S. Pat. No. 5,665,563

U.S. Pat. No. 5,492,817

U.S. Pat. No. 5,747,253 to Ecker et al., issued May 5, 1998.

Afshar et al., Current Opinions in Biotechnology 10:59-63 (1999).

Allen et al., Virology, 209:327-336 (1995).

Anderson et al., Meth. Enzymol. 101: 635 (1983).

Anderson et al., Biochem. Biophys. Res. Commun. 194: 876-884 (1993).

Aronov et al., J. Mol. Neurosci. 12: 131-145 (1999).

Berman, Current Opinion in Biotechnology 10:76-80 (1999).

Bost et al., Proc. Natl. Acad. Sci, USA, 82:1372-1375 (1985).

Bosshart et al., J. Cell. Biol. 126:1157-1172 (1994).

Bretscher, Nature 220:1088-1091 (1968).

Burke et al., J. Mol. Biol., 264:650-666 (1996).

Chen et al. Analyt. Biochem. 227: 168-175 (1995).

Chu et al., Biochemistry, 32:4756-4760 (1993).

Chu et al., Molecular and Cellular Biology, 14:207-213 (1994).

Connolly et al., Nucl. Acids Res. 27:1182-1189 (1999).

Costello, Current Opinion in Biotechnology 10:22-28 (1999).

Derossi, et al., J. Biol. Chem. 269: 10444-10450 (1994).

Ecker and Crooke, Biotechnology, 13:351-360 (1995).

Fawell et al., Proc. Natl. Acad. Sci. USA 91: 664-668 (1994).

Gerdes et al., Ann. Rev. Genet. 31:1-31 (1997).

Goossen and Hentze, Mol. Cell. Biol. 12:1959-1966 (1992).

Grayling, et al., Extremophiles 1: 79-88 (1997).

Gupta et al., BioTechniques 27:328-334 (1999).

Hanes and Pluckthun, Curr. Top. Microbiol. Immuno. 243:107-122 (1999).

Hubbard, Current Opinion in Biotechnology, 8:696-700 (1997).

Hulen et al., Biochimie 59:179-188 (1977).

Innis et al., PCR Strategies, Academic Press, San Diego (1995).

Jellinek et al., Proc. Natl. Acad. Sci, USA 90:11227-11231 (1993).

Jermutus et al., Current Opinion in Biotechnology, 9:534-548 (1998).

Johnson et al., J. Immunol. 129:2357-2358 (1982).

Keifer, Current Opinion in Biotechnology 10:34-41 (1999).

Kim et al., J. Immunol. 159: 1666-1668 (1997).

Klausner and Hartford, Science 246:870-872 (1989).

Kleinberg and Wanke, Amer. J. Health-Syst. Pharm., 52:1323-1336 (1995).

Kohn et al., Brain Res. Mol Brain Res. 36: 240-250 (1996).

Koloteva et al., J. Biol. Chem. 272:16531-16539 (1997).

Kozak, Mol. Cell. Biol. 9:5134-5142 (1989).

Kozak, J. Biol. Chem. 266:19867-19870 (1991).

Kozak, Biochimie 76:815-821 (1994).

Lam, Anti-Cancer Drug Design, 12:145-167 (1997).

Larrick and Burck, Gene Therapy, Elsevier, New York (1991).

Li, et al. Microbiol. 145: 1-2 (1999).

Mattheakis et al., Methods in Enzymology, 267:195-207 (1996).

Moenner et al., FEBS Lett. 443: 303-7 (1999).

Moore, Current Opinion in Biotechnology 10:54-58 (1999).

Morgan et al., J Mol Biol 26: 477-497.

Munson, Methods in Enzymology, 92:543-550 (_).

Myers, Pharmaceutical Biotechnology, Current Opinion in Biotechnology 8:701-707 (1997).

Palzkill et al., Gene 221:79-83 (1998).

Paraskeva et al., Mol. Cell. Biol. 19:807-816 (1999).

Pasqualini and Ruoslahti, Nature 380:364-368 (1996).

Pinkert, Transgenic Animal Technology, Academic Press, San Diego (1994).

Ricker and Kaji, Nucleic Acids Res. 19:6573-6578 (1991).

Roberts, Current Opinion in Biotechnology 10:42-47 (1999).

Ruvolo et al., Proteins 9: 120-34 (1991).

Saenger and Heinemann, Protein-Nucleic Acid Interaction, CDC Press, Boca Raton (1989).

Salas and Bollum, J. Biol. Chem. 243:1012-1015 (1968).

Seligman et al., J. of Biol. Chem., 254:9943-9946 (1979).

Sepetov et al., Proc. Natl. Acad. Sci. 92:5426-5430 (1995).

Smith et al., J. of Immunology, 138:7-9 (1987).

Smith and Petrenko, Chem Rev 97:391-410 (1997).

Spada and Pluckthun, Nat Med 3:694-696 (1997).

Spirin et al., Science 242: 1162-1164 (1988).

Srivenugopal et al. Biochem. Biophys. Res. Commun. 137: 795-800 (1986).

Standard and Jackson, Biochimie 76:867-879 (1994).

Stripecke and Hentze, Nucleic Acids Res. 20:5555-5564 (1992).

Svitkin et al. EMBO J. 15: 7147-7155 (1996)

Teilhet et al., Gene 222:91-97 (1998).

Thorpe and Ihler, Biochemica et Biophysica Acta 336:235-239 (1974).

Tueck and Gold, Science, 249:505-510 (1990).

Tueck et al., J. Mol. Biol., 213:749-761 (1990).

Vives et al. J. Biol. Chem. 272: 16010-16017 (1997).

Vocero-Akbani et al. Nat. Med. 5: 29-33 (1999).

Watts, Current Opinion in Biotechnology 10:48-53 (1999).

Wong, Chemistry of Protein Conjugation and Cross-Linking, CRC Press, Boca Ration (1993).

Zoysa et al., Protein Expression and Purification, 13:235-242 (1998).

What is claimed is:

1. A nucleic acid molecule, comprising a nucleotide sequence that comprises:
a. a transcription regulatory region;
b. a transcription termination moiety;
c. a linking moiety; and
d. an open reading frame.

2. The nucleic acid molecule of claim 1, wherein said nucleic acid molecule comprises dsDNA, or ssDNA.

3. The nucleic acid molecule of claim 2, wherein said nucleic acid molecule comprises dsDNA.

4. The nucleic acid molecule of claim 1, further comprising a ribosome binding sequence.

5. The nucleic acid molecule of claim 4, wherein said ribosome binding sequence comprises a Shine-Dalgarno sequence, a Kozak sequence, or an IRES.

6. The nucleic acid molecule of claim 5, wherein said ribosome binding sequence comprises a Shine-Dalgarno sequence.

7. The nucleic acid molecule of claim 1, wherein said transcription regulatory region is a prokaryotic or eukaryotic promoter.

8. The nucleic acid molecule of claim 7, wherein said transcription regulatory region is a prokaryotic promoter.

9. The nucleic acid molecule of claim 1, wherein said transcription termination moiety is a DNA binding protein or a sequence having secondary structure.

10. The nucleic acid molecule of claim 9, wherein said transcription termination moiety is a DNA binding protein.

11. The nucleic acid molecule of claim 1, wherein said linking moiety is a tRNA mimetic, biotin, digoxigenin, nitrilotriacetic acid, a nucleic acid sequence, a peptide nucleic acid sequence, or a peptide sequence.

12. The nucleic acid molecule of claim 1 1, wherein said linking moiety is a tRNA mimetic.

13. The nucleic acid molecule of claim 12, wherein said tRNA mimetic is puromycin.

14. The nucleic acid molecule of claim 1, wherein said open reading frame comprises a random sequence or sequence of interest.

15. The nucleic acid molecule of claim 1, further comprising a translational start codon.

16. The nucleic acid molecule of claim 8, wherein said at least one translational start codon is dAdTdG.

17. The nucleic acid of claim 1, further comprising a spacer region.

18. The nucleic acid molecule of one of claims 2, 4, 8, 9, 11, 14, 15, or 17, wherein said nucleic acid molecule is operably linked to a polypeptide encoded by said random sequence or sequence of interest.

19. The nucleic acid molecule of one of claim 18 wherein said nucleic acid molecule is covalently linked to a polypeptide encoded by said open reading frame.

20. A vector comprising the nucleic acid molecule of one of claims 1, 2, 4, 8, 9, 11, 14, 15, or 17.

21. The vector of claim 20, wherein said vector is a plasmid, a viral vector, a liposome, a microsphere, or a linear dsDNA molecule.

22. The nucleic acid of claim 18 bound to a substance of interest.

23. The nucleic acid molecule of claim 22, wherein said substance of interest is on a solid support.

24. The nucleic acid molecule of claim 22, wherein said substance of interest is on or within a cell.

25. The nucleic acid molecule of claim 22, wherein said cell is ex vivo.

26. The nucleic acid molecule of claim 22, wherein said cell is in vivo in a subject.

27. The nucleic acid molecule of claim 22, wherein said cell is a normal cell or an abnormal cell.

28. The nucleic acid molecule of claim 25, wherein said cell is an abnormal cell.

29. The nucleic acid molecule of claim 28, wherein said abnormal cell is a neoplastic cell or a virus infected cell.

30. The nucleic acid molecule of claim 22, wherein said substance of interest is on or within an etiological agent.

31. The nucleic acid molecule of claim 30, wherein said etiological agent is selected from the group consisting of a bacteria, a spore, a virus, a parasite or a prion.

32. The nucleic acid molecule of claim 22, wherein said nucleic acid molecule is bound to a solid support.

33. The nucleic acid molecule of claim 32, wherein said solid support is a chip or array, a bead, a matrix, a solid surface or a membrane.

34. The nucleic acid molecule of claim 22, wherein said substance of interest comprises at least one organic molecule, inorganic molecule, polymer, peptide, polypeptide, nucleic acid molecule, ribozyme, lipid, carbohydrate, small molecule, biomacromolecule or drug.

35. A library of nucleic acid molecules of one of claims 1, 2, 4, 8, 9, or 11.

36. The library of nucleic acid molecules of claim 35, wherein said library comprises at least two different random sequences, at least two different sequences of interest or a combination of at least one random sequence and at least one sequence of interest.

37. A library of nucleic acid molecules of claim 22.

38. The library of nucleic acid molecules of claim 37, wherein said library comprises at least two different random sequences, at least two different sequences of interest or a combination of at least one random sequence and at least one sequence of interest.

39. The library of nucleic acid molecules of any one of claims 35 to 38, wherein said library is contacted with at least one substance of interest.

40. The library of nucleic acid molecules of claim 39, wherein said at least one substance of interest is directly or indirectly bound on a solid support or in solution.

41. The library of nucleic acid molecules of claim 39, wherein said substance of interest is on or within a cell.

42. The nucleic acid molecule of claim 41, wherein said cell is ex vivo.

43. The nucleic acid molecule of claim 41, wherein said cell is in vivo in a subject.

44. The nucleic acid molecule of claim 41, wherein said cell is a normal cell or an abnormal cell.

45. The nucleic acid molecule of claim 44, wherein said cell is an abnormal cell.

46. The nucleic acid molecule of claim 45, wherein said abnormal cell is a neoplastic cell or a virus infected cell.

47. The nucleic acid molecule of claim 39, wherein said substance of interest is on or within an etiological agent.

48. The nucleic acid molecule of claim 47, wherein said etiological agent is selected from the group consisting of a bacteria, a virus, a parasite or a prion.

49. The library of claim 41, wherein said library of nucleic acid molecules is on a solid support.

50. A method for identifying a nucleic acid molecule, comprising:
1. providing a nucleic acid molecule of claim 3, 4, 7, 9, 11, or 15;
2. transcribing said nucleic acid molecule to provide a transcription complex, wherein said complex comprises a nucleic acid operably linked to a transcribed RNA;
3. translating said transcribed RNA to form a nucleic acid-polypeptide complex, wherein said nucleic acid-polypeptide complex comprises a nucleic acid operably linked to a polypeptide that the nucleic acid encodes;
4. contacting said nucleic acid-polypeptide complex with at least one substance of interest;
5. selecting at least one complex that binds with said at least one substance of interest; and
6. identifying said nucleic acid molecule.

51. The method of claim 50, wherein said contacting comprises conditions that promote binding of said complex to said substance of interest.

52. The method of claim 50, wherein said identifying comprises amplifying said random sequence or said nucleic acid sequence of interest.

53. The method of claim 50, wherein said substance of interest is on a solid support or in solution.

54. The method of claim 50, wherein said substance of interest is on or within a cell.

55. The method of claim 50, wherein said substance of interest is on or within an etiological agent.

56. The method of claim 50, wherein said at least one complex is on a solid support.

57. A nucleic acid molecule or nucleic acid sequence identified by the method of claim 50.

58. The method of claim 50, further comprising the step of sequencing the identified random sequence or nucleic acid sequence of interest or said nucleic acid molecule of interest.

59. The method of claim 50, further comprising performing steps 1, 2, 3, 4, and 5 reiteratively.

60. The method of claim 50, further comprising performing steps 1, 2, 3, 4, 5, and 6 reiteratively.

61. A method for identifying a polypeptide encoded by a random nucleic acid sequence or nucleic acid sequence of interest or nucleic acid molecule of interest, comprising:
1) providing a nucleic acid molecule of claim 3, 4, 7, 9, 11, or 15;
2) transcribing said nucleic acid molecule to provide a transcription complex, wherein said complex comprises a nucleic acid operably linked to a transcribed RNA;
3) translating said transcribed RNA to form a nucleic acid-polypeptide complex, wherein said nucleic acid-polypeptide complex comprises a nucleic acid operably linked to a polypeptide that the nucleic acid encodes;
4) contacting said nucleic acid-polypeptide complex with at least one substance of interest;
5) selecting at least one complex that binds with said at least one substance of interest; and
6) identifying said nucleic acid molecule.

62. The method of claim 61, wherein said contacting comprises conditions that promote binding of said transcript to said substance of interest.

63. The method of claim 61, wherein said identifying comprises amplifying said random sequence or nucleic acid sequence of interest or nucleic acid molecule of interest or nucleic acid molecule of interest.

64. The method of claim 61, wherein said substance of interest is on a solid support or in solution.

65. The method of claim 61, wherein said substance of interest is on or within a cell.

66. The method of claim 61, wherein said substance of interest is on or within an etiological agent.

67. A polypeptide identified by the method of claim 61.

68. The method of claim 61, further comprising the step of sequencing said identified polyeptide.

69. The method of claim 61, further comprising performing steps 1, 2, 3, 4, and 5 reiteratively.

70. The method of claim 61, further comprising performing steps 1, 2, 3, 4, 5, and 6 reiteratively.

71. A polypeptide identified by the method of claim 61.

72. The method of claim 61, further comprising the step of sequencing the identified polypeptide.

73. A method for identifying a test compound, comprising:
a) contacting a target with a complex that comprises:
1) a linking moiety;
2) a nucleic acid molecule comprising an open reading frame; and
3) a polypeptide encoded by said open reading frame;
wherein said linking moiety is operably linked to said polypeptide;
b) identifying polypeptides bound with said target;
c) determining the structure of said polypeptide; and
d) identifying moieties that have structures that have space filling shapes that are similar to at least a portion of said polypeptide.

74. A test compound identified by method of claim 73.

75. A pharmaceutical composition identified by a method of claim 73.

76. A method for identifying a target, comprising:
a) contacting a substance of interest with a complex that comprises:
1) a linking moiety;
2) a nucleic acid molecule comprising an open reading frame; and
3) a polypeptide encodied by said open reading frame;
wherein said linking moiety is operably linked to said polypeptide; and
b) identifying targets that bind with said complex.

77. A target identified by the method of claim 76.

78. A pharmaceutical target identified by the method of claim 76.

79. A nucleic acid molecule, comprising a nucleotide sequence that comprises:
a. a linking moiety; and
b. a random sequence or sequence of interest.

80. The nucleic acid molecule of claim 79, wherein said nucleic acid molecule comprises dsDNA, ssDNA, or ssRNA.

81. The nucleic acid molecule of claim 80, wherein said nucleic acid molecule comprises ssDNA.

82. The nucleic acid molecule of claim 79, further comprising a ribosome stalling sequence.

83. The nucleic acid molecule of claim 82, wherein said said ribosome stalling sequence comprises poly(dA)n or a region of strong secondary structure.

84. The nucleic acid molecule of claim 83, wherein said ribosome stalling sequence comprises poly(dA)n.

85. The nucleic acid molecule of claim 84, wherein said poly(dA)n is between five and 50 bases in length.

86. The nucleic acid molecule of claim 79, further comprising at least one translational start codon.

87. The nucleic acid molecule of claim 86, wherein said at least one translational start codon is dAdTdG.

88. The nucleic acid of claim 79, further comprising a translational control sequence.

89. The nucleic acid of claim 88, wherein said translational control sequence is a Shine-Dalgarno sequence.

90. The nucleic acid of claim 79, further comprising a spacer region.

91. The nucleic acid of claim 79, wherein said linking moiety is a tRNA mimetic.

92. The nucleic acid of claim 91, wherein said linking moiety is puromycin.

93. The nucleic acid molecule of one of claims 80, 82, 86, 88, or 92, wherein said nucleic acid molecule is operably linked to a polypeptide encoded by said random sequence or sequence of interest.

94. The nucleic acid molecule of one of claim 93 wherein said nucleic acid molecule is covalently linked to a polypeptide encoded by said open reading frame.

95. A vector comprising the nucleic acid molecule of one of claims 80, 82, 86, 88, or 92.

96. The vector of claim 95, wherein said vector is a plasmid, a viral vector, a liposome, a microsphere, or a linear dsDNA molecule.

97. The nucleic acid of claim 93 bound to a substance of interest.

98. The nucleic acid molecule of claim 97, wherein said substance of interest is on a solid support.

99. The nucleic acid molecule of claim 98, wherein said substance of interest is on or within a cell.

100. The nucleic acid molecule of claim 99, wherein said cell is ex vivo.

101. The nucleic acid molecule of claim 99, wherein said cell is in vivo in a subject.

102. The nucleic acid molecule of claim 99, wherein said cell is a normal cell or an abnormal cell.

103. The nucleic acid molecule of claim 102, wherein said cell is an abnormal cell.

104. The nucleic acid molecule of claim 103, wherein said abnormal cell is a neoplastic cell or a virus infected cell.

105. The nucleic acid molecule of claim 97, wherein said substance of interest is on or within an etiological agent.

106. The nucleic acid molecule of claim 105, wherein said etiological agent is selected from the group consisting of a bacteria, a spore, a virus, a parasite or a prion.

107. The nucleic acid molecule of claim 97, wherein said nucleic acid molecule is bound to a solid support.

108. The nucleic acid molecule of claim 107, wherein said solid support is a chip or array, a bead, a matrix, a solid surface or a membrane.

109. The nucleic acid molecule of claim 97, wherein said substance of interest comprises at least one organic molecule, inorganic molecule, polymer, peptide, polypeptide, nucleic acid molecule, ribozyme, lipid, carbohydrate, small molecule, biomacromolecule or drug.

110. A library of nucleic acid molecules of one of claims 80, 82, 86, or 88.

111. The library of nucleic acid molecules of claim 110, wherein said library comprises at least two different random sequences, at least two different sequences of interest or a combination of at least one random sequence and at least one sequence of interest.

112. A library of nucleic acid molecules of claim 97.

113. The library of nucleic acid molecules of claim 112, wherein said library comprises at least two different random sequences, at least two different sequences of interest or a combination of at least one random sequence and at least one sequence of interest.

114. The library of nucleic acid molecules of any one of claims 110 to 113, wherein said library is contacted with at least one substance of interest.

115. The library of nucleic acid molecules of claim 114, wherein said at least one substance of interest is directly or indirectly bound on a solid support or in solution.

116. The library of nucleic acid molecules of claim 114, wherein said substance of interest is on or within a cell.

117. The nucleic acid molecule of claim 116, wherein said cell is ex vivo.

118. The nucleic acid molecule of claim 116, wherein said cell is in vivo in a subject.

119. The nucleic acid molecule of claim 116, wherein said cell is a normal cell or an abnormal cell.

120. The nucleic acid molecule of claim 119, wherein said cell is an abnormal cell.

121. The nucleic acid molecule of claim 120, wherein said abnormal cell is a neoplastic cell or a virus infected cell.

122. The nucleic acid molecule of claim 114, wherein said substance of interest is on or within an etiological agent.

123. The nucleic acid molecule of claim 122, wherein said etiological agent is selected from the group consisting of a bacteria, a virus, a parasite or a prion.

124. The library of claim 116, wherein said library of nucleic acid molecules is on a solid support.

125. A method for identifying a nucleic acid molecule or sequence, comprising:
1) providing at least one nucleic acid molecule of claim 80, 82, 86, or 88;
2) translating said nucleic acid molecule to provide at least one complex, wherein said complex comprises a polypeptide operably linked to a random sequence or a nucleic acid sequence of interest;
3) contacting said at least one complex with at least one substance of interest;
4) selecting at least one complex that binds with said at least one substance of interest; and
5) identifying said random sequence or said nucleic acid sequence of interest.

126. The method of claim 125, wherein said contacting comprises conditions that promote binding of said complex to said substance of interest.

127. The method of claim 125, wherein said identifying comprises amplifying said random sequence or said nucleic acid sequence of interest.

128. The method of claim 125, wherein said substance of interest is on a solid support or in solution.

129. The method of claim 125, wherein said substance of interest is on or within a cell.

130. The method of claim 125, wherein said substance of interest is on or within an etiological agent.

131. The method of claim 125, wherein said at least one complex is on a solid support.

132. A nucleic acid molecule comprising a random sequence or nucleic acid sequence or nucleic acid molecule identified by the method of claim 125.

133. The method of claim 125, further comprising the step of sequencing the identified random sequence or nucleic acid sequence of interest or said nucleic acid molecule of interest.

134. The method of claim 125, further comprising performing steps 1, 2, 3 and 4 reiteratively.

135. The method of claim 125, further comprising performing steps 1, 2, 3, 4, and 5 reiteratively.

136. A method for identifying a polypeptide encoded by a random nucleic acid sequence or nucleic acid sequence of interest or nucleic acid molecule of interest, comprising:
1. providing at least one nucleic acid molecule of claims 80, 82, 86, or 88;
2. translating said nucleic acid molecule to provide at least one complex, wherein said complex comprises a polypeptide operably linked to a random sequence or a nucleic acid sequence of interest or a nucleic acid molecule of interest;
3. contacting said at least one complex with at least one substance of interest;
4. selecting at least one complex that binds with said at least one substance of interest; and
5. identifying said polypeptide in said complex.

137. The method of claim 136, wherein said contacting comprises conditions that promote binding of said transcript to said substance of interest.

138. The method of claim 136, wherein said identifying comprises amplifying said random sequence or nucleic acid sequence of interest or nucleic acid molecule of interest or nucleic acid molecule of interest.

139. The method of claim 136, wherein said substance of interest is on a solid support or in solution.

140. The method of claim 136, wherein said substance of interest is on or within a cell.

141. The method of claim 136, wherein said substance of interest is on or within an etiological agent.

142. A polypeptide identified by the method of claim 136.

143. The method of claim 136, further comprising the step of sequencing said identified polyeptide.

144. The method of claim 136, further comprising performing steps 1, 2, 3 and 4 reiteratively.

145. The method of claim 136, further comprising performing steps 1, 2, 3, 4 and 5 reiteratively.

146. A polypeptide identified by the method of claim 136.

147. The method of claim 136, further comprising the step of sequencing the identified polypeptide.

148. A method for identifying a test compound, comprising:
a) contacting a target with a complex that comprises:
1) a linking moiety;
2) a random sequence or a sequence of interest that encodes a polypeptide; and
3) a polypeptide encoded by said random sequence or sequence of interest;
wherein said linking moiety is operably linked to said polypeptide;
b) identifying polypeptides bound with said target;
c) determining the structure of said polypeptide; and
d) identifying moieties that have structures that have space filling shapes that are similar to at least a portion of said polypeptide.

149. A test compound identified by method of claim 148.

150. A pharmaceutical composition identified by a method of claim 148.

151. A method for identifying a target, comprising:
a) contacting a substance of interest with a complex that comprises:
1) a linking moeity; and
2) a random sequence or sequence of interest; and
3) a polypeptide encoded by said random sequence or sequence of interest;
wherein said linking moiety is operably linked to said polypeptide; and
b) identifying targets that bind with said complex.

152. A target identified by the method of claim 151.

153. A pharmaceutical target identified by the method of claim 151.
